SEQUENCE LISTING (20 sequences)

SEQ ID NO: 1 - DNA, 18178 bp, Synechocystis sp.
gccaaagggaagaaattgaggatttagctcaactagacattgtgcaagccaaaacaagtcaggaatttgcgccccaaagtaacatcagtcggggagaatttgcccgttggttatttcaggccaataacgtcttttttgctaaccagcccagtaaacaaatccgccccgctcccagtaacgccacccctcttttcaccgacgttccccccacccatcccgactttgctcaaattcaaggtttagccgacgccggcttaatcccctctagcctcaccaacgaccccactgctagtcagtttcgccccgatgcgcccctaaccagggaagatttagtccgttggaaagtgcccctagatcaacgccgagccctgcccaatgcgagcctggaaaatatcaaagaaacctggggttttcaggacgcggccaaaatcaatcccaacatttggcccgccctagccgccgacttccaaaacggcgatcaagccaacctgaaaagagtgtttggttacatcactatctttcaaccccagcgtcccgtaactaggggagaagcggcctctgccctctggtttttaggagtccaaaccgacggcttaagtgcgaaagatgtaatcaatgctcccgcatccccatcccccggagttgagcccactgttactccagatactgttaatccggatactcaaggtaattcacctaattaattaagagctaaaatcgctcacactggttaattgaacttaaatttgactaaatttaactagctttgaccatggcagtaaccatgggttaaacctatccaactaataattgtaatttcgtcggggcattcaacgccgattaagtcgccccaatagttgtcaaactgcccacaaaaactcttggcatggactttttttctttgtccggtcaagataatattagtctgctgatcagagaactgaacccctaagagttgacaatgcccctatcctcccaaccggccatcttaattattggtggtgcagaagataaagttcacggccgtgaaattttgcaaaccttttggtcgcgttccggtggcaacgacgccattattggcatcattccatctgcatctcgggagcccctactgattggggagagatatcaaaccatctttagcgacatgggggtcaaggagttaaaagtcttggacatccgtgaccgtgcccagggggatgacagtggctaccgattgtttgtggaacagtgtacaggtattttcatgaccgggggggatcaattgcgtctctgtggcctgttggcggacactcccctaatggaccgcattcgtcagcgggtacataacggggaaataagcttagcgggcaccagtgcgggggcggcagtaatggggcatcacatgatcgccgggggcagtagcggtgaatggcccaatcgagcgctggtggatatggcggtggggctgggcattgtgccggaaattgtggtggatcagcactttcacaatcgtaatcgcatggcccggctgttgagtgccatctctacccatcctgagctgctgggtttgggtattgatgaagatacctgcgccatgtttgagcgggacggttccgttaaggtaattggccaaggcacagtctcctttgtcgatgccagggacatgagctacactaacgccgctttggtgggggccaatgccccattgagtctccataatttgcggctcaatatccttgtccatggggaggtttatcatcaggtaaaacagcgggcctttccccgggtaacctagccatgacccaaggcaggtaaggttccctgaccctgggggctgactcggatcaattgttcacagcgaacagtttcagtcagaggcctagcttgagaatgatagtcggcgtgttgcgcttcttgcattggttcttgactgacacagatcatgaaaattcttaaaacccttaccctccgcggccctaactactggagcattcgacgcaagaagttaattgtaatgcgcctcgacctggaggatttggcggagcgaccctccaacagtattcctggtttttatgaagggttgatcagggtgttgccttctcttgtagaacatttttgctccccaggacaccggggcggctttttggcaagggtgcgggagggaacctatatgggccatatagtcgagcatgtggccctggaattacaggagttggtggggatgacggcggggtttggtcgcaccagagaaacctccaccccaggcatttataacgttgtttatgaatacgtagatgaacaggcaggacgttacgctggccgagccgcggtgcgcttgtgtcgttccttggtggatacgggggattattccctgacagaattggaaaaggatctggaagatttacgggatttgggcgctaactcagccctggggcccagcacagaaaccattgtcactgaagccgatgcccgtaaaattccctggatgctgctcagtgccagggccatggtgcaattgggttatggggttcaccaacagagaattcaggccacccttagttcccattccggcattctgggggttgaactggcctgtgataaggaaggtacaaaaaccattctccaagatgcgggcattcccgttccccggggcaccactatccaatattttgatgatttggaagaagccatcaatgatgtcgggggttatccagtggtaattaaacccctggacggtaaccatggtcgggggatcaccatcaatgtgcgccattgggaagaggcgatcgccgcctatgatctggccgctgaggagtccaaaagtcgttccattattgtggagcgttactacgagggcagtgaccaccgggtcttggtagtcaatggcaagctagtggcggtggcggaaagaattccagcccatgtaaccggggatggcacttccaccatcactgaattaattgataaaactaatcaggatcccaatcgcggcgatggccatgccaacatcctcactaaaatcgtggtcaataaaacggcgatcgatgtgatggaacgccagggttataacctagacagtgttttacctaaggatgaagtggtttacctgcgggccaccgctaacctcagcacgggaggcatagccattgaccgcaccgatgatattcacccggaaaatatctggttgatggaaagggtagccaaggtcattggcttggacattgccggcattgacgtggtgacctccgacatcagcaagcccctacgggaaaccaacggagtaattgtggaagttaacgccgctccaggttttcgtatgcatgtggcccccagccagggtctgccccgtaacgtggccgcaccagtattggatatgctctttccctccggtacccccagccgcattcccattttggccgtgacggggactaatggtaaaaccaccaccacccgcctcttggctcacatttatcggcaaacgggcaaaacggtgggttacaccagcaccgatgccatttatatcaacgaatactgcgtggaaaaaggcgataatacggggccccagagtgccgcagtaattttgcgggatcctacggtggaggtggcagtgttagaaacggcccggggaggcattttacgggcaggtcttgcttttgatacctgtgacgtgggggtagtgctcaacgtggcggcggatcacctcggtttgggagacatcgacaccatcgaacaaatggccaaagttaagtccgtcattgcagaggtggtggaccccagtggttatgcggtgctcaatgcggatgatcctttagtagcggctatggcagacaaagtgaaagccaaagtggcctatttctccatgaaccccgataatcctgttatccaaaaccacatacggcggaatggcattgcggcggtgtatgagtctggttatgtgtccattttggaaggctcttggactctgcgggtggaagaggccacattgattcccatgaccatgggggggatggcaccgtttatgattgccaatgccctggcagcctgtctggcggcttttgttaatggcttagatgttgaagttattcgccaaggagtaaggactttcaccacctcagcggaacagacccctggaaggatgaatttgttcaatctgggtcggtaccatgcactggtggattacgcccataacccggcgggctatcgggccgtgggggattttgtcaaaaactggcacggtcaacgctttggtgtggtgggaggccctggcgatcgccgagatagtgatctaattgaactgggacaaattgcggcccaggtatttgaccggatcattgtcaaagaagatgatgataaacggggccggagtgggggagaaacagcggatttaattgttaaaggcattttgcaggagaatcccggcgcggcctatgaggtaattttggacgaaactgtagcgttgaataaggctttagatcaggtagaggaaaagggtttggtggtagtgttccccgaaagtgtgagcaaagccattgagttaattaaagcccgtaaacccattggttaaaggttttcttcccccttgctggagtcattgctatggacaccacctaccaaaaaagtaaccttgcctgggaattgcaacagttaacccaaaatggacgggaatggctggcttggcagaccgatcgcctgtggcagggagtgccgccgataccagggctggacattcccaattgggagtggtgggaagagttaattttccacggcttactctggttaattattgccttggtgatcgccgctatttggtttaaccgcaaaacttggcagagatattggcagaatctgagcaacgttgatctcaatccccatttttcgcccccaaatcccggagttaaccattggctcaaagctgctcagcaggccgcccgccaaggagattattaccaagcctgccgatatttgtacttggccacgttgcaaatgttgcaggagcaaagaggtttagaaccgacaccggggagaactgaccgggaatatcaacagttaacggaacaatttccccaggccggcgcctacgacaccattttcaccgtccatcaggaactctgttttggccagcaacaggcttccctagagttatttgaagattgccaaggggcgatcgcatctttggaacgggctccgaaaacaaggacggatcagcgaaaatttgctgggtcttgatcccggcggtggcgcactggaatgggttctccttcgctgtcccccactaaactggcggtggtttccaaagcttgacgaagatgtttagctgcataaatggacatatccctgggcacattaatccgatcccgcagataacgcaatgcttggtctgtaccctggggtgccgcacatttttcccccgtggccagtagggtcaacgcacagcgcagagcttgatcaaacttttccccctggttatgggcattgaccttaataatattttcgtggtgtttcaccaccctggcgatcgcctcttgcttaacattttccggttccgggcaggtggcatcgggtaaaccaaaccaagggccgttgtccacccgctgttgattctgctgggcctggacactaccgttggagtaacaaccacccatttggggcggcaaatcatgggcatgggtatggaaatcactctgggtacccctataggtcatccgggtttccagcgccgcaaaccagtcttttaagcggggattatcctcccttagggaataacctttgtaataaaacaagctcgcattcatgcgctccacgtagggaacaaaaacgatgtcagcagtgctaaattctggtaagaaatacgggccaggtaatttttccaatgctttttccacttgattcaccacctgttgaaactgttgttgattccgttgatcagcaccgggaaacatggcagggtagcagagccaattacaccaggctcgaaacagtaacctttctagttttctaaggggtaataccaatggatcttccatcgcccaaagcaatggtccaaaagctttttctaaggctattaaaatatcatcgctttcagtgtaaaatctaccatccagttccaaggcaggcaacattcccgatggcactaattttttataccaatcctccttttccccatagcaaaacatggtcacctttttgattcggtaaggaatttgcttttcttctaaccataaccaaactttttgacaataggggcaccaggcatggttatcgcgataaagagtaacttgtaaagactcctttgattgtccaaataatctcagagttgcctgggcattggtgggaccattaaccgtatccacctgatagtcggatagttcagctaaagcttgccaagaaaggggagtaatcataaaaattattatcttaataatggtggaacaaaataagaaatttgggagaaatattttgtgttttaactatagcaaaaagtgagttattcgagacactttcaacttcaaaacgtttattccgagaaggtttcagtgactgttggttctaaccgtctcgatcttcgctatagtttctaattcaaccaaaatgatcggagtggttctaggacagcgttcacatctttaattctaactattagtatgatcaaaaaatcagccaagtaatgaaatcgggctcttttcgcgaaattgtattatttttacgactggtaggttttgatgtaaggtgaagcgaaagcatcggtagcaacaatatgaagcaaattattcccgcactgattactttatctttttctccaatggcgatcgccgctttgccccctcaatatcagaacgtcaaagatttggaggcaatggtcaactatgttaaagagaatcctgacgtggcggcaacgttgaaatccatagacctagaaaatcaaaccatcaactacggtcaagactgtcaggtaacgtttgagcgaaaaccttcccccaagccccttggctgggctggccccgccgaactgcttcaattcaaagccattaattgtccaagggaataattcattgccaaacggcgatcgcccgtaaaatttggatatattttacctaagcactccacaagtatttatgcaccaaatatcaatgaagcaaactcgttttgtccaagttctcatagtgtcaatattcctagttttcggaatggcgatcggactgcagcagttatctgcacagccaaagatgacgctttcaagaatccctctagaagacaagttgtatgagcctgcaggaatggcatttagacctaatcaatccaatcaactgtgggtagtcaatcatggcaatgactcaatcgcaactttaacaaatcttggagagtctaacctagaagtatcagttaaaagtgatgcctacgctgagcattttctagccaaaccaaccggcattgcatttggttatgatgacacgttcgcagtctcaaatgactcgatcaacgagttacggggaatgaatttcatcaaaaatcctgagcgaaataaaaattttgttaataaccatttcatgggtcctactttgttctcggcaagcacctatgctttggctggacaaactaagaaatatctagaagattggccgcaacctgggaacggacatgatccagaccgtgatttgactctttcacaaggatgtcctttaacttattggaattctagcgttcagcaatgttactggccgcgtgaagggagccacttggatatgttgcatgaaagtccgttagccatgggaattgcccatgacaagggtaatgcctatttcttgctagatggctgtggtagtcgagacagcaataagcagtgcttgggtaatgggcatttaatgcgctatgactttaaccgagatcaccaggaaggtaatggttttcatggcgatggcattgtttggcgttacccagaaattgatttcaaacgtgacaaagatatacctagcggaatgttggtgctagatgattggctctacttttccaatactggtatgggcaaagttgagagaatagctactacggagggaaatgtacaaaccattgtcaaaagttggtcagatcaatacgataaagaacaagttggcactggaataattagctgggaagatgttaaaagtgtctcaatggatggagatgaaccagaggctattagtaactgggtaagaactcatggagatcttgctaagatagcggaacttggccaagcttggattcctcctatggaagttcttgcagaatattcttatgtttacaatgtgcctcaggaaacttttatttcagaaaattggctagacaaaccttcgggacttgctagcgatggaaaatctcttttcgttgccgactacagctctgggtggattgccgctttcgatttgaaaactgcaaaattggaatggagatatcagtcaaagcttaatagtatttcagggctggcatttaatcctaataaatctcatgaactgtatatttcggatcgtgatactaattctctttaccagttgaagtggaactaaaatcacaagagatagaaagctcttggcgatacggatgcctgatcgtaagcactccaaactcaaacagtttgcgaagtctaggggaatcagtgtgaataagttggttgaggagctttcaaccattactttgactgaatttgatgcttataatcattttcggttaatggcagcctggggtaacatcacagaaggtttaaggcttttgggtaattttagatggattcgattagacccttagaatttgggtatgtttgacaatctgtgcaaatttctggccgaatcattttccgaagactatgctgcctggttgctgggacgaccaatcaaattgaccaagctcagtccaacggaattatccctggagccaattcgggctgactccctaattttggagcaatcggaagacttggttttacatttggaattccaaacggaacctgaccccaccatgggttttcggatgttggactatcgggtaagggtttatcgccgtttcccccagaaaacgatgcaccaattcgttatctacctgaaacgtagtaacaatgatttagtttatcaggatagttttcaggtgggggagactttgcaccgttaccaagcgattcggctttgggagcaaccatcggaagcatttctccaaagtccagggttgttgcccctagcggtattgacgcagacctctgaccctacattaaaattgagggaagtggcgactgtattggaacagattgaggataatcgagttaaagcaaatctcatggcagcaacttctgtttttggaggaatcctgctggcccctgagctgatcaaaacgattttaaggagtgaaattatgaaggaatccgctgtctatcaagaaattttacgggaaggggaacaacgaggtttacttaagggcaaactggaagggaaactagagggtcggctcgagggaaaactggaaactatacctctgttgaaaaaattaggtcttacgatcgccgaaattgccaaggagttagatattgatgttgaactagttaatcagtttgtcgctaaccaaaataattaaggatttatagtgattatcctttggcatataggctgacccgcccatcacttgccgatgcaaaagcggctaaaaatacggtctaagactgattctgttaccgtttcccctgtcacttgcccaagggcattgatagccaaacgtaaatcaatagtccaaaaatctaggggtaactgttccgccatggtttgttgcaattgcttgagcgccagttgagcttccgttaaaacagcctcttgcctttggttaatggcaaagtccaaattttgcggtgataaattggtttgatttacttgctcaataatggcattttccaaagcttcaatgcctagattagcggcggctgcagttaacactgtgttggtgatttctggtggataactgaccaaatcagcccggcctaaatcaattttgttaatgactaaaatcaaagggcggtctttgacttgctcataaatcaattgatccgcttctgtccaaccctggtgggcatccaccgtcagcaaaactaaatccgcctgttgggccgctttgcgagagcgttccactccgatttgctccacctgatccgctgtttcccgtattcctgccgtatccaatacctggatgggaatgccctctactacaagttgggattccaccacgtccctcgtcgtgcccggtagatctgtgacgatcgcccggtcggtgcggctccaggcattgagcagactagattttcccacgttgggctgtccgacaatggccacttttaaacccgtgcgcaataattccccccgctgggctgtgtttaaaatgtcctccaattgttggtacaaattctgcaattgctgacgaattgaatcttcatccaggggcggtaaatcgtcttcaaaatcaatgcgtgcttccacttccgccaaaatatctaaacaggtgttgcgcaaatcccgaatcggttgggctaatttgccctgtaatcccgccaaggcgatcgccgctgcctggggagattgggcccccaccaattcactaatgctttccgcttgggttaaatccaaccgaccattgagaaaggcccggaaactaaattcccccggctgggctagcctggccccctgttgcaaacacagttgtaaaacctgttgcacgggcataatgccgccatggcactggaattccaccacgtcttctttcgtgtaggaacggggcgctagcatcaataggagtagggcttcatcgatcgccgctttggtttggggatggcgcacatggccgtacaaaatgcgatgactttcccaagtttggttgcccggtgcatcgaataaagttttggcaatggtgagggattgtggccccgatagtcgcaccaccccgatgcttccctgctggggcacaatggccgtggcgatcgccgcaatagtatcttctaattgcattgcctagtgtcaaacaaaattttggattccatcggcttaattttaaggttttcgttactacggcaggtactacaaatccccaacagtggttaaggatcaaggcggttagccagaatggccacgggcaccatgggaatcataggtagtaaacagattgcccactgttgccagtccatgggagcagttttgaacaatacattcataaatggcagttgactgaaaccaatttgtaaggcgatcgccaccgcaatgcccagtaacagtatggaggctttggtaatggtttgacgtttacccgtcacatagcccagaaaactacgccctaactggctaatgctgagtagataaatcaccctagcggccaccagggcttggatggccatagtacgagctagggctaagtcgtcataggtgcgattaacccactcgaacatgccaaaaattagaatccaattgaacagggataccagtaaaatgcggtgcagtaactttttggtaattaggggttcgttgggattgcggggagcctgttgcataatgcccggtgatttggcttcaaaggctaggggcacagtcatggtgatggagttgatcatgtttaaccacaacacctgcagggaaagaatgggcaaattcaacgccagtaaaacgctgatcaaaatggtcatggactccccgccattaaccggcagcaaaaaggcgatcgccttacggagattttggtaaacggtgcgtccttcttccaccgccgcttcaatggaggcaaaattatcgtccgtgagcaacatatcggaggattccctagccacctctgtgccccctttacccatggcaatgccaatgtcagcccgttttaaagctggggcatcgttcaccccatcccccgtcatggcgacaatgtgacccttctcctgtagagcttccaccagttgtaatttttgtgctggagctaccctggcaaacacgcaactatcttcagcggcttgggccaattctgccggacccatggttgccaactgtcgtccttcaaaagcaatgccatccccttcagcggcaatacccatccgtttggcgatcgcctgagcggtgctgatgtggtcgccggtgatcattttcacttcaatgccagcgtcatggcaggcgtggactgcggcaatggcctccggtcgtggcgggtcgatcatgccctgtaaacctaaaaatattagccctgtttcaatatccccgtggtcaatcgcatggtgatggggctccacggtttttttagcaaaggccaacactcttagaccctgttgggccatatcctcaacattttcctcaatttcccctcggtcgatgctaaccatttgaccatcatcgaggagcatggattcacaccgttgtagtaaggactccactgaacctttgacgtaaattgttctgccgtccccatcgtggagagttgccatgtattgataatccgattcaaagggaatggaatctaaacggggtttttgacttgccaaaccggcttgactgaaaccagccttggccgccgaagctagcagtgctccctccgtgggatcccccaccaccgcccaatcatccccccgatgttccagttgggaatcgttacagagcatgcccgtcagcaaacattcttccaacactgggggtaaaccatccaacaaaacgttatccacttcttctcccatcacttgccaaaattctcctttgggactgtaaccaccgccgctaacttcgtaatgtttgccccccgcatacaccgcttgcactgtcatctgattctccgttagggtcccagttttatcggagcaaaccaccgtggcactacccaacgcctccacagcgggcagtttacggataatggcgttacgtttagccatgcgattcaccccgatcgccaaggtgacggtcaccacagcaggcaaaccttcgggaatggcactaacggccaaggccaccgccgcttcaaacatttccaaaggggaaccaccccgtccccaacccaccgcaaaggtgaaggccgccagggtaacaattacgtagagcagtgtgtggctaaatttggcaaacttacgggtcaaaggggtcatgaggctgacctgcttttccatggattgggaaatttgccccatttccgtggcattggcagtagctactaccaccccggttccctgaccaaaggtgacaaagctccccgcataggccatgtttaacctttcggccaggggcgtttcctccggtaaaagttccaccgccttttccaccggcactgcttcccccgtcaaagccgactcatccacctggagattacgcacttttaacagccttagatccgctggcaccttatcccccgaagcgagggaaacaatgtcaccgatgactaaatcctgggaaggaatccgtagattttgcccatctctcaacactgtggcttctgttgttactgccttggccaaggaggcgatcgccccttctgctttcgcttcttggatgtaaccaatgatggcgttgactagggtaactccccaaataacccaggcgttggtccaggagccgagaaaagccttaaccgtaccagcaatgagcagaatgtagagcagaggttggtgaaattgcagcagaaaacgcaaccaagctggtttgcccggtttaaatttcaattcgttgcggccatattgttcgtaacgctgggccaccgcctccgctgtcagacctaaacctggatcggtgtgcaaatctgcaagaatgtcttcccctggtcgatggtgatggaggtaagaacttaaggtggggaaatccatactgatttgggggcaaaggaaacgcccccatggtatcaactgggccgaaaagaaccaaaattactgcacgaaaaatcccccactaggcagggaggacctaaaaaaactgtcatgatcaggcaacgcactaacatcaggcgatcgccactacaaaccgacatcaatcccggcaaatttaaactctgccatggtgattaatcaatcctctcctatttcttctccaatgcaggttccactaccaaataataaatattgatgaattgaattggaagcaattacactgccactggcttttgctagaaatgttctaccttcaacgtattcgtcaatggttgtttcttctccataaacatcaaaagttacaacttttttagctcctgaataacaacttaagttgtccgttaattttggctgttttttgttgaaaacttccccttgatcaatcacacacatacccccactaaaattaacccagtgttgtccttcactgtcggaaacttttatttcatgggcgaatttatattcatcagcaaaggggccttccaccacatttttacgaaaaatttcttctcccccggaaaagcatactaaaaccggcagggtctgatctggggagttagtattggccactgccatcgcgggctgatttcccagcaggaaaatagcaatgggaagaataacaaaataaataagtaatttaatcaatttatttatcctataaactaataaaagcttggcaacaattggtttccctgcattatccttgacgcaggggcgatatggctcagcgaaatgcccacggggaccaggggagttaagcggaataaaccgatgatagcaataatatgaatcggattaaattagggcagaatttctagaaataattagtgggaagactgtaatctttttaaaaagtagcccactaaataaagggaaccacagagaacagtcaattgttcagaattttcgttggttttctctaaactttccaaggctgtaaaacaatcatcaaacagtgtaacctcttttaaatccggcataatctgccaggctaactgctccaacgttggtaaatccccgctgtcatggtccggcactggcactaataacaggcgatcgccgggacgaagtaaatgctgaaaaatttggtcatgttccttggtgctgagcatacccatcacccaggtaatggtgggcaggtaattatccgattgatcaactaacccatgggtatagtttgccagagctttggccgccgccggattgtgggccccgtctaaaaggattttttgtccgttgtagtccacccactgcaatcgtccaggccagacagctttacccatgccccgttggataacctccggtgttaatttatcccaaccctggtgttttaattcctgcaaagcggcgatcgccagggccgagttttgtagctgaaaatctcccaataggaccaggggatattcaaaaccttgccaactagcccagggttgaccatttttttccgtggcaatggcgggggtaacccaataatcggggcaatgtaattcggcaatgcgttcttgaaaaactgcctgcgcttctgggggaatgttgcctaaaaatactggcctctgggctttgagaatgccagctttttccctggcaatgtgagctacggttggccccaaaacctgccaatgttcccgacttaaagaggtaataatgctaagcaaacaaggttctgggacgttggtagcatccagtctgccccccagccccacttccatcaccgcgatgtccacctctgcctgggcaaaataaagccacaccgccgcagtaaacacctcaaacaaagtcgggcgatcagaactatccgccggggcgatcgccgttacttgttgcaatgccccgattaaactagcgtcatcaatgaaacggttatcaagccaaaccctttcccgccaatcaattaaatggggagaagtgtagcggcccaccttatagcccgcttccgccaacacactggacagataggcacacaccgatcccttgccattggtgccccccacatggacatagggcacttttgcctggggattgcccaacttggccagtaggccatggatgcgctccagacccagattaacgccggcggttttatagggttccagtagggtgttgatatccatggcaattattgttggcaaaattcagttgatgcatttaaaccgatgcggttactttggtctcggttaaatcggggacatttttcggcaatggacaggcgatcgtctcaccaccgttgtagcccactagggttgtttccccttggtaatggcggggagtttccagtaacattttccaggcttctttggcagtcaacaaattcatctgcccaggataatgggtttgtagcaaatcctgcactaaaggataataatcggaatttacgtcgggaaataagtggtgctctgtgtggtaagaaaaattaaaatgtaagcaatcaaataacttgggcatccgcaaagaaacactattaaccaaaggatcattaatatccgtcatcgggcaagccaaatggttggtgtagatataaaacatgcccatagcatgacccaggaaaatcggcaaaaagtagcctaaaataatgggaatcaattggaattgcaggtaaaacaaaatgcttaaatgcactgcccctatggctgctaattccaaccaaattctttggcggtcttgggctttaaccgtaaaagcagccggaacaaaatcggcgttaccaccagtgaagaaaagtacggaaatcaggttacgaaaattatgtacaccccaggcagtacccatgccaaaaattaaccaaagggtattaacttctcccgagggagcaaacaggtgatgaatccattttccccaagttttcggctgttcgtgcagataatttcggtcagggtcccgcaaactattggtgttattatgatgaacttgattatggagcgatcgccattgactagggggcatccaccagagggaaagacctaataaactaatcaggtaagaaattttagaacgttttttgagcacactaccgtgcatcaggtcgtggctaccgaataaaaatacggtgacgctattacccattaatagggccatgggcaaaaataaccaaagccattgcccaggccactgatggagatgacgagcgatggttagtcccgcaacataaatacctaggtttaaacctaaaataactaatttcttgggatcggcttggaatgcttccggtggcaaaagaggacggagttttttaacgtattcagattgagcagtgagcaccaggaataatccctaaaagttaaacaaagcaatttcagtgtaaatgattttacctgttttgggaaaattccgagctgatgcaaagtgttaagaaaatcttaactgcttcctttggtatatgaaaatagtcaaatctatagtcatttcaattaacgatgagagaatttaatgtaaaattatggagtgtacaaaatgaacaggtttaaacaatggcttacagtttagatttaaggcaaagggtagtagcttatatagaagctggaggaaaaataactgaggcttccaagatatataaaataggaaaagcctcgatatacagatggttaaatagagtagatttaagcccaacaaaagtagagcgtcgccataggaaattagactgggaagctctaaaaaaagacgtagaagaaaatcccgatgcaagattgatagacagagccaagaaatttggagtgaggccgagtgccgtatattacgcattaaagaaaatgaaaataaacagaaaaaaaaagaactacgttatcgagaaagaaaccgggaggaacgagttaagtactatagaatgttaagagaactaattaagctctatggtagtcaagctatagtttacatagatgaatctggattcgaagcaatccaggcttgtatttatgcctggtcaaaaaaaggaaaaaaagtttatggagatagacaaggaaaaaggggagtcagagaaaatctagtagcagggagaagaaaaggaaaaaaagatttgattgcgccgatggtttttaccgggagtttgaatgcagaaggctttgaaggatggttaaaattatatttgctaccctccctcgacattccatcaatattaataatggataatgctcctattcatcgtaaaactgccattaaagaattggctaaagaagcaggtcatgaagttctttttttgccgaaatattctcctgatttaaatgatattgagcatgactttagtgccttgaaacgagctagaatgtacgctcctattgacacgtctcttgatgaaattatccgttcttactgtggcgtttagcgtctcagctt

SEQ ID NO: 2 - DNA, 19602 bp, Synechocystis sp.
tatttgaaactactatattttcataaaactaaattcaatttcggcattttctcccatcagttccgttaataaccattggcgatcaccaactttttggtaatgttctacacgataacagaattgatcaattaataagtattcttgtaatttagggatagtccggtaaaagccaaatttctgatttttatcaaagcccgctgctgaagtggagaggacttcggcaatcaaagagggattggtcaccgccatctgactattatcagtaaactttggctcttccgcaattaccatcacatcggggtaaaaataactttcagactcagcaatccataacctcatatcgctgacaaaaatttcatattggcgatcgccaatttctagaggtaataagcggcttaaattaaaggttaagcgattatggttggcggaggctccggcaatgggaataatttctccttggataaactcacttttttcagtggcctgttcttcccgttgcagatactcagccgctgttaggattttagttgtagcaacgaccatcattgatcccctgttttatttggctgattcaagtttagctttaactttttgttactgtttaatttcaaaaaattgtccatttaagcacaggcatattttagggtaacttcgtttttgccttgctccgccaactgtgttctagaagtttaaactgctgacccagataatctaggtggggttgttgctctccataacgccaagccacataggctgtggaaatttccacaattatttctacctgctctggagttagatgttcccgcagggattgggcaaattcccctggggtttgggctcgacttttgcgaaaacccctttgctccaagtcatacagcaaagtttgataaagcctagccataggatggagttgggctaagcgacgatattgtaaccaacgtttagcctgggtccaaccaagccaacccagaaaaccagcggcgatcgccaaaatgagtccgagtaaagcgccgcccaaactagtggaaattaaattccagaaccaccgtaaaacagtgaccaggaatccggtaacagcagcccagacattagccagaaagccgctcaccggagaaggcaaccaaccggccacccaacgccagatttgctgaagcacgctaaaggtttcgttatcttcgatggagggaggatagagttcgtggccaggaatggggtcgaacataaaccaacccagccgggggaaaagtacctccgtcaaggcgtaggcatcggtattatggaccaaataataaccagtgaaagggttaaactgtccgggagcaaagcccacggttaatcgagcgggaatgtctaaactccgcaacataatggtcaacactgtagcaaagtggtccggcaaccccccttcgtattcaaacaaaaaggcgctgaccaaatcttcatcgtcttcaaaaaatggtaaatcagtgcgaatgcggtaattttgcttgagagcttgggttaggtagagggaaatttcatagggggtttctaggggccgttctgatttagctaacagttcttgcgctttaacccgcaccgccgatttgatatcggggggaacttggagcaattgggggtcaattttcacttccgggggaatgcggtactcctgcaacctagtgcgatctcgcaccggcacttctgaaatcactgtgtaggtaatgtcttttagcaaacccaccggcgatcgtaaactgccattgctgtctatgccaacttccctggtgggaaagtataaaaaccggggatggtaaagactgggaattacgttgggtaaatcctccacagcggtataggtttgcaccacattttccgtttccaggaaggagtcaatgttatccagcacaaaacgataggaccacggcgatcgttccactgtggtgatcttctcgttatcactaatttcccaaccctggcccgtatagcggtcaaaagataacgctttccaaaaacctggcgcttgggaacgtacccgcagaactaactgtttggtcatttccccccgtaaattttgattcatacggctgttgaaaccgtagtagaaggtgtcgtccacctggccgggttctgaaccatctccctctccgccggtaccagtgccatttcctcgacggtaaccagggttgttgatatcttggttttgcccctcaaaagtttgttgttgcatttccccaggactagagacgggaaaactttgcagtctataaccgggaaagcgaggcatcagagcaaagagtaacaaaccgaaggccagggtgaccgctaacagagcgaataactttttcggtactaagggggaatattccgtccgctccgatcgcctagctttctctgcacttttccagaacggtagggaaaatggaatttcttctagccccaaacgagaacggtaatccaacaccaaagtgggaatagctacggcgagaaaaagcaataaccagggggcaaaagcgagagtttgggataaagtccccgccactcctaaaaggatgagcccaatcaccatggaatagcctaaatctttccgctgaggcaaatcaaaactgtgcaatacctgtagttgcaccaataatcccgctagggccacctggctagcattgagattttccaccaagttgcccaaaaagacaaacaacatggccaccatggcgatcgccagcagaaatttgagggcaatattcttcttttttctttgacgccaactccaccaagagccaatgacactgaggggaatggcccataaactccaggtggtttgggccgctacgtctgtggccacaatgcccacaatcaccattaactgcaccaaaatgcggaaaacaatcgattcctccgttttagactcgggcaagttttgcagacgggcttgccattgctctagccaacgggccatggggaaggagaaagtcatggatggatagaaaggagtttgatgttaacgaaattgcgttttgtgagacacaaacgagagtgatcaagattttgtgtaaagaggagaattaaagtgaaaagcgttcaggaaaaagaacagcaaaatgggaagcagcggctcgccaatctcgtatagggcgctgccattttttagtgatgtttatcattgctgaatatgatagcttaaaaacagaatcttcaccgggaaaaacggattttattttgataatcttgcaaaaagggctatttaaggattcaatcgcattggtagtgtaaataacgcggcttatctccttaggatagtcaaaaatggaaatgacatggtcccaatgacgtaaccagatttgactgatggctgggtagaagttatcccatttctgagaaaaagcatccagagccgtttctgatcctgactaaaaaaaatggacattagagaagagccaaaaatcaaggagaatgtcccaatgagtcaaaaacgaagacgaacatttacagacgagaagatgtggcaactccggcgacagtgccagcggcaattgatgcgccccccatagcagtgtcggtcaaatcactcaattcatcctttttcgactgatgattatgagctttttgtcttttcaacttctctatttcaagctccagtattcttttttgccaaatgagacgttctttttccctattactacccatgacattttccccggtaaatctatgagcattataatcgcttaaaggtctttagtatgccaagtaaaaaaagatgtttttcggtctttgggccgatgacttttacctgccaaagattttgttaccgttagaagtgaatttgatcaacaatcgacaaataaggacgatcgccaaaggctatgttagaaagcaaacttgaaaatctgcggcaattctttggggaactagaccgggctttaattgcctattccggtggagtggatagtaccctagtggccaaggtagcctacgacgtgttggggcaaaatgccgtggctatcaccgccgtttcaccctccctgttaccggaagaattggaagatgcccaggcccaagctcaatggattggcattgcccatgaactggtgcaaaccaacgaaatggctaaccctaattacactgccaacccggaaaatcgttgttatttttgcaaaagtgaactccacgacacccttcagcccttagctttggccctgggttacaactacgttatcgatggagtcaatggagatgatctacgggattatcgccccggtatccaagcagctaaggaacggggtgggcgatcgccattggccgaattgcaaattagcaagttggaagtgcggcaaatttcccagttattggggttgccctggtgggataaaccagcccaaccttgtttaagctcccgctttccctacggcgaagaaatcacagtggagaaattacagcgggtcggccgggcagaaatttatctgcgtcgtctcggctataaccaagtccgggtgcgatcggaacaaaatttggcccgcattgaattgcccccggaacagatccaacagtttgtacaggatgtgtccctgggggaattagtgcaaacttttcaaaactttggtttcctttacgtcaccctagacctagaaggttaccaaagcggcaagctaaatcgggttttgaccaaataaccaaaaccaacggcatcccagcctagttctttggaacaggggaaagcccaaggctaaacccagccctgcagtttataaataaccagccctaaatattccttcagggccatggtggtcatttgcagacttttggcatcgggcatcagaccaaggatgacgttggcaaggttgaaattatttctgcccaaaggttgactcaaaaagtcggtggcggcgggaatgacaatgatacccagtttttcaaatactcttttggccctgggcatatggtaaccagacgtgaccaaaataatcgtgtttataccccgttgctggagaattttctgggtaaaaaccgcattttgcctggtattaagggatttctcttctttgatgatagcttccctaggcacgtccaaccgttgcaacaatagggccatatcctccgcttccgaggtggccactgtgcccaaccaaggaatccttccccccgtggtaataatgtagggagctttgccctcttggtagatttgtgcgccgtagataacccgatcgccagcttcggccacttcgggaaattgccggggataggtggcctggtaaagaccgccccccaaaacaacaataactggggcttgggggaaatcttccataggcagatattgatactccaaagtccgtagcagggcatcgctaacccagctattagcactgactagtaaaatagccagcgccaccagggccagccactgcaaacccaactgctgtcgccaactccaaaaaataattcccaacagcagtaaggaaattagccccaggggatagaggaataggggtaaaagtttggagagaaaaacgaacataactgggcaaaatgccaactaaatgacaatttctatcgattttgcctaatcaattagggaaaagagcatttcccccccacaaaccagttgtccatcgaccctagcttccccttgcattttggcaatgcgttgcagtttaaaggattgtaattccactgtcataattagctgatccccaggcaccactggccgccggaaccgcaccccatcaatgccagcaaaggcgaagaatttaccccgcatccccggcagttgggtcaaaatcactccccccacctgggccatggattccacaattaacacccccggcataatcgggcgatcgggaatatggccgggaaaaaacggttcgttgatggtgacattctttaggccaacggcgcatttacccggttgaaaatcaatgatgcggtccaccaaggcgaaggggtaacggtggggcagtaaatcactaatttcctggatagtaaattgggtttggacaccggcttcattgccattactatcgcttaaagtgggagtcacctccggagttgacattgccatgggaaaacttttgctaaaaatctaggacaaacgaaaatttttgaaaaactttggcaaaccaggttattcccagcaacgcttagttaaggtccagcactttggccaggttctaggcccgatcgccaccgaaaaccagtttaaagcccattgggatcccccctgggccatgagcccagaaaaattggccaattcttcttccgggttggcccactaccgataagatgggagggttgttcattccccaaacaatccgccaaatcctgtggaaatctttgtttatcacacgccggaattaacaccagatcaatccttgcctgactgtgccgtggttattgatgtcctacgggctacgaccacgatcgccactgccttaaaagttggcgcagaagcggttcaggtttttagctcccttgatgatttgatggccaccagtgaatcctggccgggggaaaaacgtctccgggcgggggaaaggggcggggccgccgtggcgggctacgatttgggtaattctcccttagattgtacgtcggagttgatggcggggaaaagattgtttctttccaccaccaatggcactagggctttgcagagggtgaaggattgtcctcagttggtcacagcttctttggttaaccggggagcggcggtggattacctagcccaaacccagcccaaaacagtgtggctattgggttccggttgggaagggggttattccctagaagatacggtctgtgctggggcgatcgccagcttgctccgggaaaaaggcattgatttcaccgtgggcaatgatgaagtggtggcggcccaaagtttgtaccgccaatggcggagtgatttactgaatttgtttaaacaagccagccatggccaacggctactcaagcttgaccgcctagaggatttgcgctactgcgccacagaggatttaattgatattctgcccaaacaagtgtcgccaggggtgttgaccgctgcttaaattcgagaattgtctttatatttaagttttgagttctcttctcctaatcattttcctagctatcattgctaaaacactatggacacctttgacgctatttaccaacggcgatcggttaagcattttgaccctgaccatcgtttaacggccgaagaagaaagaaagttgctcgaagcggccatccaagctcccactagttttaacatccaacattggcgattcttgatcattcgggatccccaactgcggcaaaccatccgggaaaaatatggcaatcaggcccaaatgaccgacgcttccctgttaattttggtagcggcggatgttaacgcttgggataaagaccccgcccgttactggcgcaatgccccccgggaagtggccaattatcttgttggggcgatcgcctctttctatggtggcaaaccacagttgcaacgggatgaagcccaacggtccattggcatggccatgcaaaatttgatgttggcggccaaggccatgggctatgatagctgtcccatgatcggctttgacctccaaaaggtagccgagttggtcaaattacccgctgactatgccattggccccatggtggcgatcggtaaacgcaccgaagatgcccgggcaaaaggcggtcaaactcccctggaagaattggtctgggaaaactcctttgcctaactaaggtttggtgtttagcaatttagtgttgagtggctaattgctgaacccgctccgactcccatttaacgcttacttctcacccctaactcctgctgttcaacgaaattttgcttgattaacacctctatgcgtactcattattgcggcgacctacggacaacccatctgggggagacagtgaccctctacggttgggtcgatcgccgtcgggatcatggtggcgtaattttccttgacctgcgcgatcgccagggcatagtgcaaatcgccagtagtcctgaccaaacccccgcttcctatccagtggcggaaggattacgtaatgaatacgtggtgcaagttaccggtgtggtcagtaaacgtcccccggaatcattgaacgaaaaaattcccaccggggaaattgagatctatgccgacagcattatcttgctcaacggcgtcaatcaacaattaccttttgtggtgtctagtcacgaagcagaacaggtgcgggaagatgtgcgccttaaataccgttatctagatctgcggcgggctaggttgagtcaaaatctacaactgcggcatcaagtggtcaaagctatgcgccgtttcctggaagatcaagagaattttctggaaattgaaacccccattctcacccgttccacccccgaaggggccagggattatcttgttccctcccgagttaacccaggggaatggtacgctctgccccaatcgccccaactgtttaagcaattattgatggtggcggggatggatcgttattatcaaattgcccgttgttttcgggatgaagatttaagagccgatcgccaaccggaatttacccagttggatatggaaatgagctttatgggccaggaggaaattcttgacctcaacgaagcgttaatttgccacattttcaaagttgttaaaaatatcgatttgccccggccatttccccgattgacctatcaagaatcaatggctaagtatggcaatgatcgcccggatacccgttttggtttggaattggtggacgtttccgacctgctgggtaacacaggttttaaggttttttccgctgccgtcagtagtggggggtccatcaaagctatccgggtgccggggggcaatgagactatttccaatgtgcggattaaacccggcggagatttgttcaaagaagccacggaagcgggcgctaagggcattgcctacattcgggtgcgggataacggagaaatcgacaccattggggctattaaagagaacctagatgaggcccaggtaaaaacgttactgcaattaacccaagctgaagccggagacttactcctgtttggtgccggagacactgccacggtggataaatccctttcccgcctccggttggtgctgggagaacagttgggtttgatcgatcccgatgccattaacctgctttggatcaccgacttccccatgttcgagtggaatagtgatgaaaaacgctttgaagctctgcaccatcctttcactgcgccccaccccgatgatttaggcgatctgaaaactgcccgggcccaggcctacgatttggtcatgaacggcgtggaaattggcggcggtagtttgcggatttaccaacgggaagtacaggaaaaagtttttgccaccatcggtttatcccccgaagaagcccacgaaaaatttggtttcctattggatgcgttcgagtatggcacccctccccacggcggcattgcctacggtttagaccggttagtgatgttgttggctaaagaagattccattcgggatgtgattgctttccccaaaacccaacaggccagttgtctgctcacggaagcaccagcggcggtggataaaaaacaactcaaggaattgtccgtggcttctacttacgtccctaaagttaaagtggacgactaggtagccctgttttttattggcccccatgatggaacgtttttcccctactacggctttatggaaaaaattgagcctctttgaccgcaccgtgggggcgatcgccatcgttttggttcttctaattgcggtggtgctattacggggcaatcaaagtcccctgatggtaacgcaatacagttggcgcaacaccaacatcggggcccaaacccaggccctgaccatgacttttaaccatcccgttgacccggacgtaatggagtcaggcttggtaattgacccacccctggcgggcaaaaccatttggcaggggcggcgttggttctacaccctaacggacatcccccgttatggcactaactatcaattgaccttacctttacctgccatggtgcggggtccaaggggacaggatttcaccagtctgatcaccagtcgggcccgggctttggcctatattggtgtggccggcgaagaaaggggaaggttaattctctacaacatcaccaacccccaacaaccccaaaaaattatcctcacccctaaggacctcaccgttcgacagtttcaaatttatccccaaggcgatcgcctgattttcaccgccaccgatccgacccgtcgggggggacaacagaatattttcactgtcgccaccggtattaataacctcaacacccaaactaaagttctaccaggcaaattggaaaggctttgggaagaccaggattatgataaccagcgcattgccctggctgctaacggcagtatgttagttattgctagagaaaatgctcaaaattctgctgactctggtttgtgggtagcccccagtggagaaaatcccagacctctaggtattagggccgaaaaatttattgttggacccaatggtaatttccttgcagtgggccaggaggggggagtgggtttaattcccctaattccggaggccggggcgccccattttttggaagcaatggagcaagccttggatttttctgctgatggcaaacaattgctattaagccaacaaaacccagactttagccattccctaattgtttacgacctgcaggaacgcactcaaagggaagttttacggggtccctaccccctaataagctgtcgttttgacccccgctcccaacccagggcctattgcttgcaggcggattttatccaacgggaaggcgagcctgtgcaaccagaaccctacctggcggtgattaatttaaccgacggtcaagcccgccccctcttagctttacccaattaccccgaagttgatctcaccgttgctcccgatggtctggggttaatgtttgaccaggtagccacaggacctactcttggttcccatgatcttctcaccaattctcaaagatccattgtggatggacgagtttggttgttgcccctacccggagatttggccacggaaaccgatgcggatcccgatccccaagaattgaccgctggttttgctccccgttggatgccataggctctgatccccactggattagggttattaggctgtgctgacgtccaaagggtcagattatttggtacgctaatttaaacagtttgggaatagtaagtaagcgtgaacattacgattggacggggaaaaacagcccgtcgagcctatggtatcgacgaaattgcactggttcccggagtgcggaccttagacccagccctggccgatacccgctggaaagtgggcgccattgagcgggaaatccccattattgccagcgccatggatggggttgtggacagccgcatggcagtactactatccgaactaggcgctttgggggtagtcaatttagagggcattcaaacccgctacgaagatcccaatccgattttggaccgcatcgcttcggtgggcaaaacggaatttgtggggttaatgcaggaactctacgccgaacccatcaagccggagttaattaccaagcgcattcaagaaatccaagccgctgggggcattgccgcagttagtctgactccggtgggggcatctaaatatgccagtacggtggccgaagctggggcggatttgttatttatccaggccacagtggtctccacggcccacctatcccccgaatcggtggagagtttggacctggtcaagctttgtcaggaaatgcccatgccggtggtgttgggcaattgcgttacctacgaagtcagcctggagttgatgcgggccggtgcagcggcggtgctggtgggtattggccccggtgcggcctgtacttcccggggagttttgggagtaggggtacctcaaccgacggcgatcgccgattgtgcagcagccagagatgactatttgcaagaaacgggtcgttacgtgcctgtcattgccgatgggggcatcattactggtggggacatctgtaaatgtattgcctgcggtgctgatgcagtgatgattggttcccccattgccagggcggcggaagctcccggccgtggtttccattggggtatggccacccccagtcccgtattaccccggggtactcgcatcaacgtcggtaccactggcaccattcgggaaattttagtagggcccgccaaattggatgacggcacccataatctactgggggcaataaaaactagcatgggcaccctaggagccaaagatatgaaagagatgcaacaggtggatgtggtcattgccccttccctgctcacggagggtaaggtttaccaaaaagcccaacagttgggtatgggtaagtaattcttgccattgcaactatcaaccccctgggattattctggggggatttttattgggtttatttgttaccctggtctgggccttgttgaccaatgccccaccactacccgctattaccccgccgctgtgaaacactggaaaacttacctagttttagtcctattggcgatcgccatgttgttaccgttgctgtggttggtagctacggcgttgaaggccccgacggaggatgtctttgggggtttgggacaattgttaccccggcaacccaacctggggaattttatcaaggtttggcaagactatccctttggccgttacctattcaacagcgtggtggtgtccagcatcactgtggcccttaacttgtttttttgcgccctggccgcctatcccctggcccgtttaaactttgtcggtcgggacatgaccctagcggcgatcgtggccacgttgatgattcctttccagttaatcatgattcccctctacattctggcagtgaatttagggctcaggaatagctacgtgggcattatcttccccagccttgcttctgcctttggtatttttctgctccggcaagctttccaaggggtgcccaaggaattggaagaagcgggccgcatcgatggttgtcgagaactgggtatttggtggcacattatgattccggccattcgccctgccctggtaaccctagctatttttgtttttatcggtgcctggagcgattttctctggccactgattgtgctcgatcaaccagaatattacacattgcctttgggcgttgctcaattatctagtgctttggattttgactggcggctcattgcagcggcttccgtgattgctattgctcccattatcgtactatttctgtttgtgcaacgttatatcatcccctccgaggccagcagtggagtcaaagggtagagccattttcttcggtaaaagttagtccagcatagtcaattaaccggtgaattaaatcctgaccaaagctagccgcaggggtccagaaaccgccttggagagatgatttgggtctgtctttggctaaacataatgcggcttgggctaatatttttgccgtggaaccatagccggggtcttgatcccctgtaactttgaccatggtggattggccagaggcagtttctccccaaaagcgtaaatcataaaagccctgctgttgagcttcttcagtggggccttcccctggtttaggtaggatttggagtaacaaccatcgactggggccgatcgccaaagccaaggccataagatccaaactccatttaatgccctgggctgctaaccaaccgattatccctggccccgttaagatgccttcctcatattggaacccttcaccatagacttcgttttggagggtattggaacgtaaaacaatgggagtgttgatccctgccatgacaaagggggcagtccatccggaaaagatggggtcattttgcacggggatgaggctggggggatggtcagaccggtgatcaggtttaaagcaaaggatgtaaggattgcggagggcctggcgggtggaaggatcggcgatcgcctcttgaatcaggttgatgccactggcgatcgtgcctccggaaatgccaccttggcaagctttaacccccattttgacccggatgcaaggttccccccaacgccgttgactttgttgttgtaagtaatacaccccgaggtcagaaggaatggaatcgaagccgcaacaatggacaatgcgagcccctgattgcttggcgatcgcctcatatttttggatcatttgttgtacccattggacttccccagtcagatcgcagtaatctgtgccggtaatggcacaaacccggactagggtttctccataaagggcataggggccgactgtggaaaccacaactttggtttgggagcacagtgtagtgacagcggcttcgtctttgatatcaaccacaaaggtgggcaaatgggcagcttgagggccaagggattgaaccaaacggtcaagtttagcttgggaacggccggcgatcgcccagttttttgctgctgcagtttcccagtgggacaacaaatagcggcaaacaatcccaccgacaaatcccgtcgcacccacaattatcaggtcatatggcaatggcttcgtcatagagtttaacggtagttgaaaggttgaatgggatacaaagctgactatgggatagtctggattgatatactgacggtcgagtcttaaatttgcctaatgttatttgcccatgaggcttccaggatgatgtgcccccagaaatggtttaaacgattattacccatggttttgggtgtttgctgtttgctaaccggctgtgtggattatgacgtgggcattcgttttccagagcaacattatggggaaattgtccaacacattaccctgggggaacaactaactaccctgagccaagcagaagccgatcgttggctcaaaagtttagaccagcgggctaaggacttaaacggtcacacggatcgcccgtcggaggaaacccttgtgataaccattccttttggtagcggccaggagttggtggaaaagttcaatcgcttttttaatccccaaccccccaaaggaaggcgatcgccagagccagaccagttggatttgcttaatttgaaagcggaactcgcactagagcaaagtaattggttattagcagaccgcaatcatctcaagttaaccgttgacctgcgggccctaggggttttatccaaccaaggcaacattatccttagtcccggctccctaattaacctcaattttgatttgcaaactcccttaactctccaaactcccaatgctccgggggaagaattaggcacagaagtgcctgggcaatggcagttaaaacctggacaaattaacgttattgaaacgaccttttttgtgcctagttatttggcgatcgggacaattttaattatcatcctttgtttagggggtttttatctcaaatatggccgttggcccggagtgcttgatagcaaagttgagggagtttaagaacgtgtttgaaaagtccccctaatccccccaaaatttggagagaaactgagtaaaagttaccctcgtaccgccggagcgttagtaaggagatttagggggcggaataaaattttccaaatacgttctaaatccttaaactttaatcaaaaattgtcaggttgccattagcgccctagggttaactcctcttttttaaccaaaatgccccggggtcgccataccattaacaccattaaaattagcccgatgaccataacccgtaaagctccagcttgactctggtctaaaaaagcaatttggggcaataaaaacctggtgagggaatcataggcccaaaaaataatggtgcctaacaccgtgcctacattactgtcagctccccccagcaccacaataattcaagcgttaaaaatcaacaacgtgtcaaaattacccgggtaaatactggtcaaatgccaagcaaataatgcccccaccaaactggcgatcgccataatagttgtctcccaaggagaactgtttttgacagaagatccaacccgatggagatgatagtttcgtaaatatgttttaggtctgatgtcgcaaatcactctttccagataaggttagttgctcctctgatcccaatgagttgatagcaaggaagttttcattattgtggcgatacaaaccaactagacttgatctaccaccacagtctgattttcatcgtcttccggatttggccccgagcgtttgcgccattcctgggccaaaatgccatacttgggtgcaccaatcatggctattacgaatagcgttgtcaggataaccacaatcatgcccccggtcgaaacatcaaagtgatagctcaaataggtgccaagcacacaggacagtacactgctgacgaccgagtaccaaagcatatgatcaaagcgatcgctgagcagatagccaatggatcctggagtgaccagcatggaaattactaaaataatgcccgctgtttgtaaagccgccacaatggtgagggccagaaccgacaacagcgtgtaatacatcacttgggtgcgtagaccgatcgccttggcatggtttggatcaaaacaaaacaagagcaggtccttgcggcgcaacaaaatcacgattagggtaatggagccagcaatcaaggtttggatgatgtcttgttgagaaatgcccagcacattgccaaacagaatatggaataggtcaacattactaggaattttggtcaccaacactaatccaagggcaaaaaatcccgtaaagacaatgccaatgactgcatcctcctttaaccgggttttagacttgacgtaaccgatcgccaccgttgccccaaagccaaaggtaaaagctccaatggcaaagggaatattcaaggcataggccagcactacaccggggaccactgcatgggaaatggcatcccccattaaggaccaacctttgagagtgatgtagcaagagagcaccgcacacactagtcccacaaaggcgctcacccaaattgcccggattaaaaattcatattgcaaaggctccactaaccaatgccaaaagggaaaggccaccactaactgattcacgcatctacctctgtcgattcaaacatctgattcagactcagcattggtagcccaccaaaagtcagttccaaattttctttggtgaaggtttcttcagttttaccctgggccaaaatagtgcggttgagcagaatggtgtggtcacaaaaggtggaaatggaggccaaatcatgggtagaaatcagaatggtgtgcccctcatcccgcagttccatgagtaaatcaatcatgcctttttctgttttcacatcaaccccggtaaaaggctcatctaacagaattaccttgccttcctgggccaaggcccgggccaaaaatgcccgcttcttttgaccgccggaaagttccccaatttggcgatcgcggtatttcaccatgcccacccgctctagactttccatcaccaaacgacggtctttggcgctaggaatacgcaacacattcatatagccatagcgccccatcatcaccacatcaaacacactgaccggaaagttccagtccacctcatcagcttggggaacgtaggccatcaactgttgcttttgggccttttgtacggaaaagccaccaatgcgtacccgtccctgactcggttgcaaaaaccccatgatggatttgaacagagtcgatttaccactgccattgggccccaccagggcggtaatggtgccgggttcaacggtgcaggtagcgttataaagggccaaacgggcgttgttataggtaacgctgacaccatcgacggatatatcaagacgagatagggtggctgccatggcaaaagaggggttcagattttaggtagtggtaaatcgtcaagattaagtccagttgattacagaaaaactcaacgtgtcaaaacacaaagttgttttcattgctgggcattggtaccggccaaaagaccgttggtaatcacccgggcatcatattccagcaaatctaaaaaggtgggcacagggccttcctctgtggaaagggagtcaacatagagattgccgccaaagcgagcgccggtggcctgggctacctgtttttgccccttatcgctgacggtactttcacaaaagattgttggtacattattggttttcacctcctcaatcacggtttgcacttgcttgggggtaaactgctgttcggcattaattggccacatgtaaatctcctccataccgtaatctctggccaaataggaaaacgctccttcgcaactcaccaaaaaccgctggttggcgggcacttgttccaaatccgctcccagttggcgatcgatcgcctttaactgctcgctgtagactgctgcattggcgttgtaatatttggcattgtccgggtcaagttcgacgaaggcttggcggatattttccacatagaccagggcattacggggcgacatccaggcgtggggattgggcttatcggtataggggccgtcggcgatcggaatcggttcgatgccttccgttagcaccacagagggcacatccttcacgttgcccaaaaattgctcgaaccaacgctctaggttcatgccgttgtaaaggattaagtctgcgtcctgagccttgacaatgtcactgggagtcggctcataaccgtggatctctgcaccaatgcgagtgatggattccaccactaacttgtctccggcaacattctggaccatgtcggccaatacggtgaaggtggttaaaaccttctttttctcctctgtttccccctggacttctgtggttacggctgtcacttcttcgctgggagcattggatgtcgtgacttccgccgtgccacaaccggtcagccaaaaggcgatcgccaaaccagacgccagcagtcctccccgggaagcaaatgatgttgccatagtgataatggtctttcatgatcatctcatttttcattatcttctcataattgcagtgaaaacggggagttaaaatttcttaacttttaccaactggtaagctcgtgggctttttctgaccactaaaactttctgctttttacatgctcagaacggggtgtgcctggcgacttgtaccccacgattttcctaaatggcgaacggtctatggttatttccagccatggcacgaagatggtacctggaaaaaattaaaccgtatcttccgtgagaaagttcggctcaaagctggaagaaatactcaccctagtgccggttgtttagattcacagtcactcaaaagggcttgaactggccaagaacctggctatgacggtggaaaaaaggtcaatggtcgtaaacgtacaatccttgtggataccatgggattaattctcgatgttgtcgtccatggtgcccatcgctccgaccatcagggtctgattctacttggcacctggtttgctcccctttggcagtgcctgcaagttatatggactgacagtacctttggcggaaaggattttatccattgggcggagcatacttttggttggaccttgaatgtggtcagcaaaaagtagggacaaaaaggctttgaagtcctaccccgccgttgggca

SEQ ID NO: 3 - DNA, 24303 bp, Synechocystis sp.
tggaagtgctctgccatgacatcaacgtccatattccccaccacctctccgttgccatcccttcctataacctacgactagcccacggaagtttaaaagaaaactggggaccttttctttacgagcgcacctttaactggcaattaatgcaacaaattagtgggcaatgtcatttatatgaccccgaacatggctaccgcaccttcggctccctgaaaaaagtttaatactgggacaactagtaatttttgacccatgattggtcagtaattaactttgactgatccccagggagagaaataccagatcacaaattaactatcttggaatgcggccatcgagctgtattccttttttcttttcttggtgaggaaaaaaactttctaagtgggcagatgggagcggttcagactagaaagatccactcggccaaaatgaatgctaaacgcaacctgcatattctccaaggttttcactagccaaggtaccccttcccgggccaccgactcataatgattaaacaaaattgaaaaacgtttttccaaataaggcttaaccttggagcagaaaagcacaatttttagcaatacgccaatggtcttcttcacaccaatattggaaattagatcaataaagacatagtgatcaggatttttgctaccttccaccaccaaaaagttgagcaactgactagaagtccgcatcattaaaaattctgttggcttctgttgattgcactctggataggcattttggaatagatcaaataatttcttatcaaaacttccttgtttgtaattattctgtagggagccccggatatattcgtaaagttcatccttaaaagtgccaaaagattgggtataggccacatggttacgaaaacttttggaaagggaccggtaactctgaccgttttccacggaaccaacaaaatgtttgagggaactgtttagatcccgatcactcagtagggtgggattttgcacgggcttgatcaattccttatccggcaagatgagtccggctttattagttctggccatgcgcacccggtaggtgacgtactgagaaaggttaatttcaaacttacgctcattttgagctttgatgcggtggatagtttggcgatgttcccgactgctgtcatcccccataagacaataatcatacagatagggatagcggtggataagggtgccgacggagctagatttcgtttcaaacttggtgctgacaacgctggccaatcttttcaagcggacaaattgttcgctcaaggtaaagtttttaactagttggcggatgttaccttttgtattacccccggggctgatgttagccactgctttttccaccatatttaccagctggggaatggaacgctgggtttggggagaaagttgccaatagttgatcagaatatagcaacaacggttaaaaaagtagttaaattcttcggtattatttttaactctaattactttttctaagtgggtagaaacttgtccttgggaactgccctgtccgctcaaaaaaagattacccattacttccaatacccgttccccttccaccgactggaccaagtcgagcagataatcataaattgcctgctcgtcattcattttgtaggggtcgctagcctggggatggttatagttcacagtggtaaccctctttggggaaaagggggcaaattgggagcaggaaaaatcgataatgaaaacgaaatcatgagataaggtcaaagacaccctgggcctttaatcaggcaccgctgtggatggtaagatacaccctggtcccatcggaataaaaccaggatgtttttactggtcattactggcttccgatttttctgtatctatttctcctccaatgtaagtgatgcaagcacccatgggaggtgatctagatcacagataaaaattgcaaatgacttaacattcctgagtcaattctaactcagttttcctccgccatatttttcactcctattctttcccatgaactccaaactttccctatttgacatccgtacattcctagaaagttgtctattgtctgttaccaatctttccgaggatatacggataaataatatttgcaccgatacaagatctttggtgtcaggggacctatttcttgccttgcggggggaaagttttgatggccatagcttcattccccaggccttgacggcgggggcgatcgccgtggtgacggatcggccagtggagggattaggggagacggtggcccaatttttagtggaagatactttggtggcctatcaacacatcgcagcgggatggcgacaacggtttaccattcccatcattggggtaaccggttctgtgggtaagaccaccaccaaagaattaattgcggcggtgttgtcccagtttggcaatgtgcataaaaccagagctaattacaacaatgaaattggggtgccgaaaactttgttggaactatccccagaccatgattttgccattgtggaaatggccatgcggggtcgggggcaaattgctcttttggcggatattgccaagcccaccatcggtttaattactaacgttggcactgcccacattggtcttttaggctcggagttggcgatcgccgaagccaagtgtgaattactagcccaccaaccgcccgagagcaccgccattctcaatcgggataatgctttgttaatggagacagcccaacggttttggcagggaaaaaccattacctatggcctagaagggggagatgtgcacgggacagtggatggggaaaatttaatccttgatggagttagtttgcctttgcccctagccggtgtccacaatgcctctaattacctagcggcgatcgccgtggcccaatgtttagggttggactggcagcagttacagtcggggctgacggtggagttacccaaggggcgggcccgtcgttaccaatggggccaggacgtggttctattggatgaaacctataacgctggcttggaatccatgttggcatccttggatttattggccaatacccctgggaagcgacgtttagcagtgctgggggcgatgaaggaactgggagattatggcccgacatttcaccaacgggtgggggccaaagtgaaagccctaggtttagacggtcttttcctattagccaatgatcccaataccgatgccattgccgccggggctaacggtgtggagacccaatccttcagcgatgggcctagtttagtggcagccctaaaaactaccctccaaccaggcgatcgcctcctgtttaaagcatccaattccgtcggtctaggggctgtggtcagtcagttgttggcagaaaatcccacttcggtttaaaacggcaaggcttgagtcagttaaaaatggggttaatctcactccagcgggatcaatcgagatcaataggtgtggggttggttggcagtggcatccaacaaatggcctaaaaccagatggttaagaacatggctaaaaacgtctatccaaaccaatctggccttatcccttcagagggttttgaggtaattcaataaatccgccatctcctggggattgggttcaaactggggcatgggaggagtttgaccactcaccacctgatgaatgatgtggctctgggaccgccgttgggataccccccaaagactggggccaatgtaaccgtcagcttggatgccatggcaaactgcacagttagcttgaaaaatggcccgacccctatccgcatctcctttgagagcgagtacctggcttacataggggtcaaagttcataaaactaaacaaagccactgccaatatcaccagggaaacaatgcctgccataatccatatcccagtgggggaagcatttaccgtggcaacagttggacttttttcgattacaggggccatgggttaagaacgattggtcaaggtggccagaaccaccactcagaaacttaatctaacttaatctattgtattgatgccgggcattcctgtctttgctcagtcccagattgaagtccggattaaccctgttgacaaactaaaactcccccgatatcgatacccagaattttcctaggtcggggggggagcattgacaaaaatctatccagctcggccccaatggatcagcaagggcccgccggcttataggggagcaacttgcacttccaccgtagcgatgacttcggggtgcagtttgatttgggcttggtaaaaaccagttttgtggatatccggcagactgatgccccggcgatcgagactttggttggtagccgcttccactgcatcggccacttcctgggtggtaacggtaccaaaaatagcttccgcttcccctacttgttttttgatcacaaatcgaccgatggtttccagggccactttgcgggcttcagcggcatctttttccgctttaagccgttctagttccttgagccttctttgttccacctggcgcaaaataccaggggtggccaccactcccagacccttgggaatgagatagttccgggcataaccgggggccacttccactaaatcgccggtgaaacctagtttattgatagtttcgtttaaaacgaccttgactcgctttgccataaccgtaatttttccaatatgaagtttgtttagcttgtggagcaattcacaaacctccatcttaacctttagggggaatcccgccaaaattggcgatcgccagggagcaatttttacctggggcaatggacagaaaggcaaagttactctgttaccatgggggacggccctccgcaaccagatgccaatgtccagtctgatttccctttttcgccaacattatccccagggcagtctctgctgtgacctgctcgaaattgaccgggggttatatatagtccaagccagcatcaccttagaaggcattgtggtggccagcgcgttagcagcccagtctcccctagaagctgctgaggatttagccaaagaaagggcgatcgccagcttggatttaacccatatttcatcaacagtcccacagtcttccccaacagccattgttgaagatatggaagcgaagccatcgcccccaccttcctcccctaaaaaagaaagtaaatctccgaagcagaaccataaagtcgttactccccccgccattgtcaatccgacaccggtaactcctgcgcatccccctactcccgttgttgaaaagtcgccggaagttgaggcggcgatcgcccctgagccaaccttaacccccgctcccatttcctttccccctagtcctgatccagtgctgtccttagaggagccgaccccgcctccggcaatggtcaatagcacctttaatcaaccggaagagtccgctcctattgactccgaacttcaactcgactttgcgaccccagaattacccctcgctgttgaagctaaaccagattcgcctgaaccggatatggcggtctccggagcaacagaactgcccgcaggacccatggacttttcggaaattattgcccgcagtaacctggaactaaaacgactgggctggaccagtgaccagggacggaactatcttttgcaaacctatggcaaacgttcccgtcagttattatccgatgagcagttaatcgagttccttgcttacctggaacaacagccagacccaaattaatcggattgatcctgcttttgtcccagggttggtaaaacataaatccccgggacattttataggcttaactagctagaactttttctgtttctgcggcgatcgccgttggactgagaactgtttcttctactttgtcctgttgttggcggctgagataatctcgcaattgttcaccaatttcctggcggacaatttcccggtattggaggaaatgttcgttatgggcgcaaagggtgatgtagtgctcaaacacggcgggattatggcgcaaaataccgaaagcatggtgccagaagcggaacctagtgtcccgtttaacgccctgccgccaaattacaatcgccaaagcctttaaatccactaaattggggagtttgaaagcggtcttgcatttcggtgcccccagcttgagaaaacaacggtaattgcggtctaaatagccatggggatcgtataactcccagaaagcctccacatactcgttggcaatgtcttccaaggggcgagtgggaataaagttcatcaacgtggtttggttgatgttgcctttgctctcatccagcaggcgattttcccgttttagtcgatgccacagggctgtgttgggtaaagcctggagcatggcaaaggtggtggtgggaatggccgtctgttccgcaaaacggacaatgcgatcgccagccccctgtttttccccatcaaaaccgatgatgaagcccgccatgggtcgcagaccagctttgatgattttatcaacggagtccgcgagggaattacgggtattttggaattttttggtgaattctaaactctcttcatcgggggtttcaatgcccaaaaagaccgcatcaaaatagcactccaccatcaaatccattaattcctggtcatcggccaaatccaccgaggcttcagtgttgaatctaaagggatattggtgctcttcctgccaaactttcagttccttgagcagtaatttaacgttgcgcttattgccgatgaaattgtcatccaccataaacaccgatcgccgccaacccaactcgtaaagataatccaattccctgagtaactgggctggttccttggtgcgaggcttacggccgtagaggacaataatgtcgcaaaattcacactggaaaggacaaccccgggaaaactgcaccgacatggaatcgtaggcatccaattccagcaggtcatagcggggaatgggggtagtggtaacgtcgggtttttcttgggcccggataatacctgctgtttccccccgctccaacgcttccacaaacaggggtagggtaatttccccttcgtccaaaatcaaaaagtctgctccggcattctgcacttcatccggtactgacgtggcaaaggggcctcccactgcgaccaacttaccgtgggccttagcttccttgatgttttccaccatgtcatcccgctggacaatcatgccggaaatgattactacttccgcccaatcccattccgcttcggtcacattgcggacattgcggtccaccagcttaaactcccattcttgagggagaatgcccgccacggtgatcaaccctaggggaggcaataaaaccttccgacccactagctccagaattttttcgtaggaccagaaagtagggggaaacaacggatagattaatagggcgcgcatggcttctcgctaagggaagatttatgttaaaaaatctaggaattgattaggtatgggctcaactttagctaaagctagcacacccttgccaggaagtccccgaaagccagaccgttggcatagataaatcctatggttaaccatggttatcccctaggccgctccctcttgtccatagctatggctatgttgggggcattgtcattggtttttcccttgcctctgttaacccaagacatggttgacagtgagcgttccctgccattgctgggggaccatcccctggaggaaaggttaattgatggcatggatgaggggatcaaaaaaaattaccggggggcgatcgccatttttacggaattaattctgctagatcctagctatgctgatgcctattttaatcggggtattgcccgggccagaatagcggactaccaaggggcgatcaccgaccacagccaggccattaccctaaacccccaattggccgaagcctacaaagctagggggaaaatttattggcaattaggagagcaagcccaagccattgctgacctagaagcggccctagcccttttctctgaccaggaaaatcttgtgagccaacgggaagcctgggaacaactgcaaacatggcatggtaatccttaagagcctgtttggaaagttttattccgccccctaaatcgacccactaaagctccggcaatactggaggactttcactcatttccccccaaatttggagggccaggggggcttttcaaacacgttcttaaataggccccaatatttcttctcaacttctccgacagtttcccaagcccgtcgatcaatttttaactccccttgcccacgccctagttttactgaattttgccgatctttttgtcgatttgagggacactttttgcccacaaatttgcatatttgccctggattcgggaccattaacttgagttaaacaaagttaagttttatggcatttttctttcctcccaaatagccttgctaagatagcgggaaagccgggttgctagtcccatggttgcttttttattgagtacaaggtaagcataaatacctaaacatgagacatttttgcgacgcttggattgaagaatggtgtgaagccaatggttggacggatttgttcatggaaagactcaattactattgggcttttccccctggggctgtcatgccggagcccattcctggcaaaattctccgggcaattaaactagaaaaaggtctttgcgaagaagaaaaagcttggttaaccgccgctgtggctgtttctgctttggcgatcggttctgcttttctgctcaaaaatcccatgcctgtagtactggcttttgcttttgacgctgtaacagcggggcgtttagagatggaagaaatttaaaaaatttgagaagattaatccattacctgcgatatcaaacactctgattggagtaactaagcaacatttatggctgattccatcaccccagactccaatgaacacaattatgctgagctggctcaacaatgccagaatttggccagccaagtccagagcctcagccaacgggtggaaaatctggaaagcaaactgaccttaatcccggacattgagcgctatggtaggttgcaggaatgcttaattgcgggggattttaagacggcggaccacgaaaccacccgcattattctcgaaaccctcaatcgtaatcgggataacctcagcccagaaactttatctgctttgccctgtacagttttaacggtaattgatcgcctgtggcgtacctacagcgaagatcgttttggctttagccggcaattggaagcctatcaaaaggtaggtggtaacaccgatactttgcgtacccaagaccggaaagtgatgggggcctttgcccaggaagtgggttgggtgcaggaaggaaaggttcgttttgatgagtatgaccaatgggatttttccctcaatgctccggtgggtagtttccctgccatttggtggaagtctccctatggtttgaaaatggtgacttttttcttcattcgtttgtttgaatgtaacggctagcccgatgccctaactacagttttttaacgcttcgatatcagccccggcaattttgtccagattgcgggtaattttctctatgtcccaatcccaccaagcaatggttaacaaagcttgaactacttcgtccttaaaacgttgacggataggacgggcgggatttccccccacaatggtgtagggtgccacatggttagtgacaacggctttggcggcaataatggccccgtctcccacctttattccgggcataattaccgcttcgtacccaatccacacatcattgccaatgactgtatctcccttatggggcggttcttcttccagttctgctgtttgccaaccattagcaaaaattgcaaagggataggtggaaaatccttccatggcatggttagccccattcataataaattttacattccgggctagggcgcagaatttgccaatgatcaggcgatcgccaacgaagggaaaatggtacagaacattgcgctcaaagttttctgaatcttcgggatcgtcgtaataggtgtaatcaccaataattatgttggggttagaaacagtatttttaataaagcaaatctgggcaaatcccgccatgggatgcttattttggggatctgggccgtacattatttgtcacccgtaacggttctggagtcgtgtctatttctctattcttaactctaggaattaaactcccatttctgccaagtctatcaaagtttcctgggccgcctcttcgtccacaatgctaatggcaaagcccacatggacaatcacgtaatcccccaccttaacctcgggtacgtaggcaaggctaatggttttaatgatgcccccaaagctaacctttcccgtcagtaacaggggatcggagttgggcattaaactgacaacctggccaggtagggctagacacatgggaggaaaggaaggaggaattggcaaataagtcagtggagctttgagaaaaaccaaacctgggccatggttcaactttaacccaacagtaaagttcccatttgggccaccaactgatgggtgacgggcaaactgtcaatcaccatggccaggcagagcaacattaggtaaaaaatggagaacttgaacaatcccctagccaaatcccgatcgccgggagcctgtttgagttgccaagctttgaccagaaattgtccccccaaaataatggcgatcgccagataaagtatgcccaactggtgcagaggataaactagcagcaaactgaaaggcaccaccaacagtgagtagtaccagatttggctaacggttttttcctcccccgcaattaccggcaacatggggacattcacctgggcataatcatccttgatcatcaaagccagggcccaaaagtgggggggagtccaaagaaaaattagcgcaaacagtacccagggagtccagcttaaatcccccgtaacagcggcccagcccaccaaggggggaatggaacccgcagccccaccgatgacaatgttttgggccgtgtgtcgtttcagccaatgggtatagaccaacatgtaaaagacaatgccggataatgctaggcaaccgcttagtacattgacaaaggtggccaaaagagcgaacgaaagtacccctaaggctagggcgaaaatcaacgcatggcggggttgcacttttccggctgggatgggtcgggccctggtgcggagcatttcatagtcaatgtcttggtcgtaaatacagttcagggtttgggctgaagcggctgcaagggttcctcccagcagagtgatcagcaatttgggtaaatccacccgaccttccgaggcaatccacatggaagcagcggtggtaatcagcaagagggggataattctcggtttggttaactgataataacttttgcacaccgcccccatggaatcgtgttggcgatggatttttgtcgaagtaaccatagagaatcaaaagaaagggtaagcaggggggagatcaaaaatttttcctgaattttaagcgagtattccccaactggacaaaaaatggcagttagtttaaccgcaacggcagaaatttccctccctagggagacttaaagatttttatcatcaggatgtaatgggggccgtcggtcgatggtcccgcaccgccaaaacggcgatcgccaacagggtagcccagagacaaattcccactgtatggtgggctacggttaggggttcaatttgtaaatggagctttagcgtagcaactcccaataggatctgtccccccaaaagggctagggccccattgcccaactggcgcaaaaagggatttagggccggagtgcgcctggtacaccaaaaagtcagtcccacccccaacaccgtcggtaacacccccaacagatgactgtgcaacacttggcaaaattgtgtgccggcgatcgccaaacactgatgggccgcccactgggaacccaccaatccccccagtaaacattgcagataaatgatggtggtgctagccagggccaccggccttaacttgcccgccgtttgattactttggtagggcatcagacagagaccaatgatcaccaaagaacccaaaaaagccatggctgtgcccaaatgggcggttacaatgtcaaaccgtagcagttcgatcactgtcagcccccccaacaatccctgcaccaggatcaacgccagggagcctaaggtagcccagggtaaccacttgggtagaacccgacggaaccaccagctcaagcccaccagaatcagggtactaaaaccaatcaacgcggcatccaagcgatggaaccattccaaaaatacctgtaaattcatttgttgactgggaacccactgaccatagcacagaggccagtcaggacaggccaaccctgcattcatcaccctggtggcactgcccaccgccatcaataacactgtggcaatgacaattttccataccagccgacggatccagacttcaacctggacaaaggcttggggttgactttgggagcgaatggcattggggagaatagactcagccatggttttgatctgcaacctcaacgatgggggcaagggggggagaaaaaccccagtcaccgtcagattctaaccaacactctccccaggggaaagaaaacttggctacagcatcaatattctttagatttacttcagatttccctgccctaggcgattgatttagttgagatcgggaaaatttggtatttaaccggttacatgtagttgcgtggaggtgagcaatacacttaagctattttatctggtgaattaaatttgaagttattcttgtggctatcatgggagtaattgcttcccatcacagctatggataaacccagttcctttttcgttgtagattctgaaggttctcaaatacttagggaaatttgcattattaatcactgtggggaagtggtatatgaagcattggtggatgaagtcttagcagatcgagcacagggtttaaaagtttttcctctgaaacaggtgttggaaaatttttggcgcatcacccatgggcatttagtcattttccattatgctgaccatgactgccgtgtcttacgaaatagctgtttgcaggtgggcattccttggcggccaataagcacccaatgtactattgagttggccaaggaaaaatatcccggcctaagcagttactctttggagtttttgagcaaaaaatttttcttaaaggttgaaggcaagtatttcaaccaagaatcggcgcaccgcgctcgctacgatgcccttttcacccaccagttatacaattttttacaagattgcaacggcttttccaaatcaatgaatattggcattaatcctttttcttccagtagagttgacaccccttttcaggaacacgttgatgcaaaagctatttatcaacaacaatttgaacaactgaaggcaatcattaaggaagtcagacaggataaaaatcgacaatctaagggtgtagtagtgacagggattcctggatcagggaaaacccacctgatgatgcggctcaccaaggaaattttacagataaaccgagtattattcatacgccaccccaacaatcaagaatcaatcatttaccatatttacagccgcattttagaatcgttcatttatcaaattcccggcactagttatagtcagctagagtttttgctggcccatagttttactaaattaattagccaaatgaaaaatattcggctgacccaaaaagatcaacatatagtcgatattgtccagcaaaaccccttaaatatttatcagctactaggaacggaaggaacagataagaaacgggattactgggatcacattgaaaaaagagccagtgaatggtggacagagcactatggcattggaggaaatagccctgaaattattaagggaattatcaaattttgtcgctatacagaacgttatcgtcaacagaccactgcacgttggttggccggacaagaattgagtgaagaagatttaatgttattgggtttgagtaattggcatgaaaacctgggtaaggagggcttttccctggaagcaatttcagttttcagtaaattatccttgttggacgagccgttagtaatggtatttgatcaattggaagtgctgggtttacccaaaaaccagagcttattaattggcttcggcgaagcgcttaaggaaatttttacccatgtgcccaactgtttggttatcctaaacctttttccagatcggtggcaatattttcagtcccattgggattcttccgtggtggaaagggtaggccaatatcacattaatcttgaccgtccagacgatcaattgatgggtcaggtgttgatgaccaaggctgatgcagtaggggttaagctcacggatatatttagtgagcaagaaattacctcccttttgcgctcaccgtcaattcgtcacttgatcaataatgcttccaaactttattctgctaaggctaatggatctggcatcatccctcagaatttcgaccttggtaacaaaaatcttgatttcgagcaaagattaaatttactggaaaatggattaagtgatttgcaagcaaaaatttacgacattggcttgactttaacaaacttttttgctcctacaagccagaatgtaagcaaagcattaattggtaatcccgtgggggaaactaatcaaaataatgaaattgacgctgatcttaatttcaattttttcattacagaaaatcagtcgcaacaatcgctagaaatacaggtaaaagatttcttctgccaaaatcgtgaaatcttaaaagatgattatcaaaaactacaaataatcactgaatccgatgatattggcaaactaaaaattattttggaagcccttaactcaatacactctttcaaaactgattttcttagtctgggcagaaaaaggttaccagaacatatcttgattcgtaccgctaaagcctcaacagtcatcgcatttttgcaccgcgatggtagtcaatttaccaactatattaagaactttaatcaattggtgtcattggagaagaggcatcaatttcacctctggcgagatcgacggcaaaaggctatcaccggcaaagttggccgggaagaaattgaaaaactcaataattctgccaacggacaatttcaatttatggaagagcgggaacgggtggactttgagctgttgtatttgttgatcacagcaatttataaccaggacatagagctggaaatagggcaggcttggtcgatcgcctgtggggaattaaggcataactggttggttcaactgttagaccatggttgaatcccggcgatgggaaccgaaatttttttctagcctttaaggctatagacacgctgccattgctcttctgtgacccgaaagttggtgctgttgggagcccgaataaccagtaaatcccgtaaaatctggtcctgtttgagttcgtggcggcggaggggttggcccagcaacttgcgtaggaatctgagctgtaccctcggtttatccaacttagcttggtcagacattagccaatattgcccatcgggtagctctgggtaaatttccggcggcttagttacttcggcaatggcataaatccctgcttctggcccggccatccagatcaaagcccgatcgccaacttgaatttctttgctataacgggtcaccaaccagggcaattcctcttgctcctcaatggcttgaagaatgcgatagtacttaggattgccttgaaataaccagtaggccatggtggggtcgcgaatcagaacaactgcggtgggaactgggccgggggaataaaattatccctctttcatcagactgagggaattaaaatggagataaaaggctgaaaagcccggagggagaatgacaaagacaagagaggcgaaaaagacagtacagtgtgtagacacatatagtgaattgtataaagatatatttccagaagtgaggtcttatgagtcatttaaatatatcattgtgggaatattaagtgatataaaaagaaagagtctacctgcaatagcatcatcactaggattgaaaaatgaacagggactactgcattttatgacagattctccttgggaattaaaagaattagaaaaaagaagattaaatattatcttagaagttttagaaggaagagaaataatagtaataatagatgaaacaggagaccccaaaaaaagggaaaacaacagattatgtaaaaagacaatacattggaaatttaggaaaaatagaaacccgacttttaatgtggggtaatagaacgcccagcaaattaataaccaaatcatcgtggggaatactgctaccagtcatagctaggatttccccgtcaatgtactcgtggcagatttcttgtttcgcttcccaggctagatattcctctggggtcaatggggtcggtcggaggtcgggcgaagcaatcatggttaatttgccgccaatttggcgatcgccattaggttgatcaacaatcacatttacattttaaattaaagacaagttttccaaaatcgaccgaatatcgccgtaaacctgtgggaaaatcccttcaatcatatttttaatcccccgtccctatgaaacgccgtcaactccttggccaatcggcgatcgccgctggaactgctgctggtttagttgcctgtggcaaggccaccacctccaataatcaagccaccgctggaggagaactgccaaatattcgttggcgcatggtcaccagttggcccaaatccctcgacactctttttgggggcgctgaggacttctgcaaggcgatcgccgccatgaccgggggtaaattccagattacgccctacgccgctggggaaattgtgccggggttagaggtgctagacgcagtacaaaatggcaccgttgaatgtggccacaccgccagttattactacatcggcaaaaatccggccctgggttttgcctgcgtgatgccctttggtttgacggcccaacaacaaaatgcatggctctatgccgggggggggttggaaatgatccaaaaagtttacagcgactttaatatcattaacttcaccgccggtaacaccggggctcagatggctggttggtttaaaaagccagtggaatccctagcggatttgcggggtttaaaaatgcggattcccggcttgggagggcaggtaatggctcaactaggggtcaacgtccaggtgattcctgggggagaactattcctagctctagaccggggcaccattgacgcagcggaatgggtagggccctacgacgacgaaaagttagggctaaataaggcggccaaatactattactatcccggttggtgggagccaggccccagcttggacgtattggtaaaccaaagccaatgggcgaaattgccgccggaataccaagctattttccaggaagcggcccgtagtgccaatctattgatgctatcccgttatgaaacttttaatagcattgccctgaaaacattactggatggaggcacaattctgaaatccttttctccagaaattttaacagtggcccaggaaacttcccgagcattgctagcagaatttgccagtaaagatgcccaatttaaggaaatttatcaacaatggcaaacattccgcagtcaaatgtttagttggaatgcaatcaatgaacttagctatactaaattcgtcgaaaaagctccctaaccgaggcaaaatctttggaaattgccttctgctaggaaagccgtaatttctgcttctgaaaggggatgactaaaccaataaccttgcatttctcggcaacctaaatcgtagagacaacgagcttggggctcctcctctataccttcagcaataatgtttaaattaaaaccattgcccaacagcagcatggcctgaataatggcagcatccttgggggtgtggaggatatctttagtaaaggaacgatcaattttcagggtattgaaaggaaaggttttaagataggaaagggaagaatagccagtgccaaaatcatccatggaaagccgaataccgtgggattgcagagcattgaggagattttgggtagctgtgacgttctgcatgacaatattttcggtaatttccacctcaagacgatggggagggagggaagactctgccaaaatacgtaaaattgttggcaacaaatccggagcttggaactgttggggagacagattaatggcaatgcgaaaatcatcgtctaccgccggagcccaatggtggaaatgttgaaaagctgtacgcataatccattcccccacgggaataattaggccagtggtttccagcaaaccaatgaataatcctggagatactaaaccgtgaatgggatgttgccaacgaatcagagcttccactccacaaaggcgaccggattgcacatcaataatcggttggtaataaagcaagaattcatccctttccaaagcctggtgtagggcatgctccaattgaattctttctaaatggtcactattcatggactcttcatagaagcagtattgccgactgccgatgtccttaacttcgtttaatgctattcccactcggttcagcaaaatttccacgttatcaccatggttgggataacaggcaatgcccgcatagccctggaggtaaaggggattattagcaatgaaaaatggtctttttagcactgccaacagtcgtcgcacaaaaacttcaatttcgtcgaggttacggcaggattgaattaacaatataaaagtatctccacgccaccggcaaagtaaatcttctaatctcacatgggcggataatctttccgtgataattttgagtacttcatcggcgacgctgtgccccagcaagtcatttaaagattgcagttctcggaaacctaaaagcactaaacctaaaacactgctttgattctgaacgttacttaggactttacccaattcttgcttgaacaaaaagcggttgggcaagtccgttaggggatcataaaaagcttggtaatgaattgattgctcaattactttacgctctgtgatatcgaaaatgtaactacgtataaactgactttcggcgggatagtgaatatgttgctcataaaatttatctcctatttgtacctctctagtgcggagatagtaaatgggagtatcttccctttggggaataatatctttgagtaggggatgggccatggaatctttctgaatggagggaaacttttccttagtagctgtgttcagataggtcaagttgccgaagtaatcgatctccaccatggggtagggactatattcaatacaggcggctagtttagatagcttttcatggctcagttgggataagtcatcctcgtggccaatcagagtgatgtcccattgttcctggggaaaatgctctgatagatctggggttaaccgctggcgatcgccgccgccggagatagtatctttggggtcggtggggatggcctgatagaaaatttgaatatcttccgatagggcgatgacgtctccatgcaccaactcataatctagatgggattcaccgttaacccacactccattacgactgcgatgaccttctaaatctccatcaatgatgtggaaggagatatctaaattaggattaatttttttgattagggtggcgtggcgacgggaaatttggggagaaggaataacaatggtgttgctcggatgacggccaatggaataacaagaattttgtaaacgaatgacctttttaaacgagggtgaatcaacgaccaattgataaagcataggagcgttacaggaaaaaagctgagcctgaagaaaaatagttttagagagtgactattgtcgcgactagggaacaattattgatcaccactccaaccagttaccactgagaggaaataaataatagaacaatctgatttgtatccgttgatgccatcatttagattccccaactaatagtagcccacacaataattattcagccactatattctttgtcctattattgtcaagctaggacaaacttttagaaaaaatctgggttttcattcggttttccgatcctaaagggggctaccaaaccatggtaaattctggggaaagttgattcacgtataaaacctatgccattcaccattgactctgcccgcggaattttccctaacaccctagcggccgacgttgtacccgctaccatcgcccgttttagccaactcaatgccgaagatcaattagctctgatttggtttgcttacctagaaatgggtaaaacccttacgatcgccgcccctggggccgccagcatgcaactggccgaaaatgccctcaaggaaattcaagccatggggcccctccaacaaacccaggccatgtgtgacttggccaaccgagctgacactcccctgtgccgcacctacgccagttggtcccccaacattaaacttggcttctggtaccgtttaggcgaactgatggagcagggctttgtcgcccccattcccgctggttaccaactttctgccaatgccaatgcagtgctagctacgatccaaggcctagaatccggtcaacaaattaccgtattgcgtaatgccgtggtggacatgggcttcaccgctggtaaggacggcaaacgcatcgctgagcccgtagttcctccccaagataccgccagccgcaccaaagtttccattgaaggggtgaccaatgccactgtcctcaactacatggacaacctcaatgccaatgacttcgacaccttaatcgaattgttcacctccgacggtgccctccaaccgcccttccaacggcccattgtcggtaaagaaaatgtgctccgctttttccgggaagagtgccaaaacctgaaattgatcccggaacggggggttactgaacccgctgaagatggtttcacccaaattaaagttaccggtaaggtgcaaaccccttggtttggtggcaacgtgggcatgaatatcgcctggcgctttctcctcaaccccgagggcaaaattttctttgtggcgatcgacctccttgcttcccccaaagaattactcaactttgctcgctagaataactcccttcagagttttgtctttgccatagtcggtcggacaagcagccaaaatactaataactgtgggcttctcgttgcagagcaggctcactttttttattatggtaatgataattacaaatcaatatcataagttaatgttacaaaccgccgaagcaccttggtcccaagctgaaacccagtctgcccatgccctgttccgtaaggcctaccaaagagaattggatggactgttggcaacggtgcaagcccaagcttcccaaattacgcagatcgatgatctttggaaactccatgattttttgagtgcaaaacgccacgaaattgatggcaagtacgacgatcgccagtcggtgattatttttgtttttgcccaactgctcaaggaaggtctggtgcaagcggaggaattgacttttttagccgccgataagcaatctaaaattaaggccctggcccggctgtgattctagtaaatatctaaaactccacaactatgttcctggggggaaatggaattcatctgttagtcatttctccgctgccttgcttgctgtaggtgtacaccgtttggggctgccattggtttagggcgtcggcggtgaggtcggcaaaggatacattttcccggtccagactgacttggagagtttccatggggtctttcccttgggcgatgaggaatgctagagcctgtaggtctggccattgtttgagctgtacgagaaaatcctttaccaaagcggggtagggagaatggtcgtgcttttgccaaggctggcccgatggtaagttttggtctaggccgacgtggaaaattaggctatttttgccaaatagggcgatcgccacggggcggttgtcttcatgcaccatgaggggggttccctgtaacagggcccggattttttccaatttttcgtaccgttgttgggccgaagcaacgttgggagtaaatttaatggcgatcgccagcagatagtttttgactaaccagtgccctagtttttctgccttggtggtcaggtaatgggagttatagtcgttggcctcaattaacaggggcacccgttcgacaatttctagaccgtagcctttcagtccggcaattttacggggattgttggtgatcaaacggatttgtttcacccctaaatcgttgagcatttgcgcccccatgccgtagtcccgcagatccgccggaaaacctaatctttcattcgcttccacggtgtcatagcccaaatcctgtagggaataggccttgagcttattgaccaaaccaataccccggccttcctggcggagataaaccaccacccccaaaccatggttttccagcattttgagggccgcctggagttgcatgcggcaatcacaacggagagaacccagggcatcccccgttaaacattctgaatgcatccgcaccatcaccggctgttgaccaaattcactaggatcacccttcacaatggcaatgtgctctgtttgatccaataaattgcggtaggcgtagagtttaaactccccaaactggctgggaaattcacagatggtttcccgttgcacaaagcggtcatgttggagtcggtaactgattaaatcggcaatgctgatcagtttgaggtcatatttgcgggcatactccaccaattccggtaggcgggccatggagccatcggcattttgaatttcgcaaattacccccgctggatagagtcctgctaggcgagaaagatcgactgccgcctcggtgtggccggctcgtttcagtactcccccagttttggcccgcagagggaaaatatggccaggtcgggaaaggtcctccgggcgggtgacggggttaatggcaatctggatcgtccgggcccggtcttcggcggaaatccctgtggttacgcccaaatggggggctgcatcaatgctgacggtgaaagcggtttggttgctgtcggtgtttttactgaccatcaagggcagatccaattcgtccaggcgatcaccagtcatggctaggcaaatcaagccccgggcttccactgccataaagttaactagagcaggggtggcaaattgggccgcacagattaaatccccttcattttctcgattttcgtcgtccaccaccaccacggccttgcccgctttaatgtcagctagggctgcatcaatggcatcaaacatcggtaaatcggacgggatacgggggacagaaacgttcaaggtgcagaaaaggaaaaagttaaaacttaagaagctggaggaaaattgacaacggtggtcgcagaagatcgactgttgcaacaatttagctttgttaactattttaaagcttttctgggaaggctttggtttcttcgtcgtaggggatgaatttctctaaaaaagggaaacttattctgccatggctatagtcctatgatggaaagcgccctggttccgctggcccccatccatgattgaccgcgaatttttgcttgaccccaattcccactccccatccgacctttcctcggctaaaagtcgcccctggttagcttccccctggccctggaccatggcgatcgccgttttgttgggttggggaggtatggccctgtggcagtccccttggtcgaagctaaaactaccttctttctccagttcggtggaaatcgcccctctagtgatggaagggggggatccctatctgcgggccttgatgcggaccatcagtgctagtgaagccaatacgccccaaccctataacgttatttacgggggacagcatgtccaggacttgagcgaacatcccaaccgttgcgtcaaaatttttatggggccgaatcggggcaattgcagtactgcctctgggcgttaccagtttttgaataccacctgggctgaaaaagcgggtaaatatcatccccagccccagaaatttttgctctggaaaaattacagctttgagccggagtaccaggaccaagtgctttaccgttggctagctgataccaaagcctgggggatggatattggggcccaattgcgggcagggcatctagaccaggttttgcgtcacctttcccccacttggactagtttgggctacggcattgaggacaatgtgattaccagacgtttaccggaactttatcaaaagttgttgcaagaagaattgcttttggcccaccatcatcaggattgaagggatttgacaaaaaat

SEQ ID NO: 4 - DNA, 13934 bp, Synechocystis sp.
ttattcctagttgtccccttagccacaaaccgcagtgtgcctgactggaatttgtctcgcttctaaaaaggttttcaactccccaatggttaactgaccatagtgcaatagggaagctaacagtgccgcttcagctttgccttccgtgaatgcttcgtacacgtgctgacaattacccgctcccccggaagcaatcaccggaatttccacccgttcggcgatcgccgctgtgagggccagatcgtaacccgcttgggtgccatccccgtccatactggtgaccagcaattcccctgccccccgtttggccacttcctccgcccaggcgatcgcatctaggccggtgttttcccgccccccccgcacatacacatcccagccgggattatccgcatctaaccgacgacgggcatcgatcgccaccacaatgcattgccgcccaaaacggtcactagctcgactaataaagtccgggtcccgcacagcagaggaattaacactcaccttatctgccccagcccggagcagatttttaatatgttccaacgtagaaatgccgccccctaccgttaggggaataaacacctcttccgcagtgcgataaaccacatcaataatggtgtcccgttgctcgtgggtagcagtgatgtctaaaaacaccaactcgtctgcccccgcttcgttataggcccgggccaactccaccggatccccggcatcttggagatcaacaaagtttatgcccttgaccacccgccctgcattgacatctaaacaaggcaaaatccgttttgctaaggtcataattttattgcaaatatttcaaacaaaaattgtggtcaatgtgaattaaacgaagcaaataaaacatccgaacccggcaatttgtccgtagtatcccaaagattaagctcgaatggggcggtgaagttccatttccccatccaagtcccttggccgacaaatacattttcccagcaacttagttgaggggaaggttttctgttgactattgatttagaaaccaaagctggggaaaggaatgggcaccggcaaactggggatagggggagagtagttcactccgccggtggaacaactcagcccgggcaaaaggttggagcacatactctgcaaggctccatccggagtgatcggaatgctgaagccattgaacaaactaccactcaaaagggcgactaactcctccggagtcatgggaatcacagaaccgaggatgccctccaaaacactgaccttttcccccgctttaacccgagttctttgctgtggattttccggatcactaaccaacaccgccccctccagaaccttaatttcccccgattgaccttcattcctttccatgacatacactgtgccagtcaccgccacactgacccccgccatacaaccattgacaggaccactggccagcaacttaccccgttccaactctacgcattgaccgacaattacgctggcattttcgctcaatcttcctgacgccccattattaaacaatattccggctctggcttttttggtcactatgacctggccaaattccgcttggtcatccacttttgcctggatttcttcgatgtaaacctggggaccgtcaatgatttcctgcaccactgcttggctgatttgggcttggctgggggccacggcccatcccagggcgagtaagggcgcagaagcaccgatggcacttagtttaagaaatttggctttgggtgagctgggtggggtaatcatggcatggcaccgggaaaggatctgtaggggaacgtaaaattggttagggggggggaaagtcgtttttctttggggctagacgacacaggaaaattttcgcttccatcttagccgtttatcaacgtattttattggagaaaaagtggtcatggcagtgcgttttttgttcaacttaacctttgcctccgtggtcacagtttgtgttttggggggcgggaaatggagttatatatctttagcccagtctcccccagaagtcatcttgaaaggagaaagtaaaactccgatcgccactgctcagcaatattttaatcagggtctcaagcattatcagcagggtaacctaacagattttcaagcggcccaaagttcctgggaagaggccttggccctctggcggcaggaaaataatttaccccagatagcggtcacgttaaattttctttgtttggttcagcgtcagttggctcccccgacaaaggccttagcctgttaccaagagttattaaccaccgcccaagccttgaaagatacccaaacggaggctaatggtctggtggcgatcgcccaattgcaattacaacagggtaactaccaggaggcatttgaaagcttggaaaagtccttacccctttggcaacaagccaactttaaaacgggggaggtgattagtcagaatgagttggggcaaatgtatttgactttgggggcaacaactcaggccgagtatttttacaacaaagctttggcccaggcccaatccttggggaaccggggtaatatcgctggcattgaacataatttggcccaggtgaaattagccagtggagatctaaaaggcgctttagccctggaagaaaatgcttttcaacattggcaaggtttgatccatgaccttggcgatcgggccacaccggatcttgtgcgaggccaggcagcgggattaaataacctcgcttttatgcaggtgcaaaatcagcaattaaatttagcccaagaaaattaccaaaaagcattaataatctggcaacggttgggggataaaaatggggaagctagtagcttaaataatttgggttacctagcttttttgcaaggggatttacccacagcgctcgattactatcaacgggctttaatcctacgacaaaccactggcgatcgcctgaaggaaagcttaactcgctattggttagcggtggcttggaagcaacagggagaagtacagcaggccatggcggaaattgaagaggcaatttatttactggaaaatctacgacaggccattggcaacgaggatttaagggctagctttttagcttccaaacaggattattatcagttttatattgacctgttaatggaacaacatcaacggcaacccaatcaaggctgggatgaaaaagctttggtggttagtgaatccgctaaagctagatctttgttggatattttggcccaaaccccagggaaaatcaccaagggaatttcttctcagttggtaaacgaaaaagaaaccattgaacagcaactcaataccctccaggaaaagaaaatcaagttatatagttctaactatgatccacaaaaacaaagggagttggaccaggccataacagatctattgcaacggtatgaccgagttttagggcaaattgagcgacaaaatcccgactatagcgcccttacccgcccccgaccattggacttacctcaaatccaagccctgttggacaatgaaaccctattgttggcatacgccctgggcaaagagcgcagtcatctctgggtggtaagtgaatctagcctgcggagttacgtacttcccggcgctgaccagttggacgaggccattaagacctttcgccaatcctttttggagcctacccagcggattcgtcgccccctggccatcaatcaaggggcccagttatatagtcagttaatcccagacccgacagtgattgcaggaaaaagattgctgattgtgcccgatggcgtgttgcaatatctgccctttggagccttggtgacggacaatcctcccggccagccccccacctatttaactgatcgccacgaattggttaccatgccgtctgcttctaccctaggcattctgcggcaaaatacggcccagaggaaaccagcccccaatttgctggcggtgtttgccgatccagtattcaccagtagtgacgaaaggttgggtacggcgatcgcccgtcgtccagacaatttgccggtggatttggaactgtccgccagggatgcgggggtttattttgaccggctcccctacaccgaacaggaagctgaacaattggtggccctctttcccccggaagccagtttaaatgaattaggctttgaagccaccagggcccgggtttttgccgacaccatggggcagtatcgttttattcactttgccacccacggcattctcaacagtaaaaatcctcaactgtcgggactggtcctttccctactcaatccagagggggaacccatcaacggattcgtgcgtttgtacgatatttttaaccttaacctgccagcggacttagtggtgctcagtgcttgccaaacaggacttggtcaggaaatccggggcgaaggactaattggcctgactagaggttttatgtatgccggggcggctagggtcgtggttagcctgtggagtgtggatgacgaagccaccgcccgtttaatgacggagttttaccgggctttactgcaacggcaaatgcccccggccgccgccttgcaatgggccaaacaaaaattgaaggaagacccccgttttgcttccccctatttttgggccggtttcaccctccagggggaatggcaaaatttacgccctactaatcagttagtcaagggaacgtctccggcttcatcggtaccgacaaaaagaatgggcacatcgctaaattgacccaaacccacggaatcaaacagttctaccaattcctgccgttgttccggcagactggccacaatcatcaacgtatcttgccaaataccttggttggcatagagattggctttgctcaaaggttcattggtttcttcaatttgctctgttatctcaacatctaattcctggcgcacgagcccggcttccaccgcactgcccaccacttggttgtcaatggtgcaattaacggcaaatgcccaagcatattccccgcctggttctaaccctacggttgtgggaacggcgatcgccaaaatactttccttactgggcaccgccacggtttggctatggattaattccccggccccatccactaggacaaattccccttccactcctttgttggcgggtacataaacaaacaaactaggattggccgtagcggtaaacgtgtcttggctgggggccactattctgaggttggtgttggcctgggtgaaacaactgccgcccctagtgcctcctcctccggagctttgggtggtaccccgatcaccagcctggggaaatttcaagctaactagggtttccgttggggttgggctaaaggataaatcatcaacaccagaggcaatggtctggggggcagatacggtaaaggttagggcagtggctaacaaaagcggcaagggatgattaaccatggcaaatttgggaatttgaggaattatggttccagcttagcctaggtcaacccggggtaacgaggggatgttcagtggcgggattgaccaataaaaattccgttggctggaggttagggggtctacagtggagtgagaattgtctaaggacgataaatccccacttttggcaccatttggggggcagggggccgataattgcgaccaaaaccgtagtcccccaaccacatcaagccatggcgaatattatccgttcccccctcggcaaaatattgccacaggcaatggactgcggtgaggggcactgtggactggttcatcagggagggatctgggcgatcgtcccccggcaagacaaataatttcagatctggtttggttaccgttagttcccccaatacttccaagccgtagggccaatagctttttccccccaacagccgcaccacaattacctctgcttggcccaatacccgatcgccgtagtcatcgagggaaaaagactgttgcaaattcagcagattacaaacccgcagatcgggaaaatccccgggcaatcggggccacaccctggcgattgcctggatatcagtgtcagcggcggttaaaaaaattagcggcgcaggagtttgctcaacgataataaccccttccatggccggggtccaaccaccgggaagactagcaagacgatgcataatagttgaaaatatatggagataaatccatggactgacaagggctagataaataatttccttaaaaaaatcttagatctttaaaaatacttagattttgaagggttaggcaaaaacctaggcatgatggaaatagaaatccatggggcttggtcgttgaatgccgccgcccagtctatgttcaattcggcatgctacccgctttttcccccattttccgtcgactcttacctgctgtcacctttgaacgcttgctccgtttttggcgtaccctggcccaacaaacaggagatggggtgcagtgttttgtgggggatttgcccagtagtttgaagcctccccctgggccatcggtgctggaggcagaggttgaccaccgttttgccctcctggttagtccagggcaatgggccctgttggaaggagaacaaatttctccccaccattatgcggtgtccatcacttttgcccagggtatcatcgaagacttcattcaaaagcaaaatttgcccgtggtagcggaagccatgccccaccggccagagactccatctggtcccaccattgccgaacaattgaccctggggctactagaaattctcaatagcgacagcaccagcttttccccagagccttctctccaggacagtttgcaggctagtcaagtcaaacttttgagccaggttattgcccaaattcgccaaagtttagacctgtcggaaatcctcaacaatgcggtgacggcagtgcaaaaatttctctttgtcgatcgcctagttatttaccaattccactacagccaaccatccctaacaccgctcgaggaaaatcaaattcccgcccccaggccccgccagcaatatggagaggtgacctacgaagcccggcgatcgccagaaattgacaccatgttgggcatcatgaccgaaaacgattgtttttcccaggtcttcagctacgaacagaagtatcttaagggcgccgtggtggcggtcagcgacattgaaaatcactactcttcttcctactgtttagtcgggctgctgcaacgctatcaggtccgggctaaattagttgctcccattattgttgaaggacaactttggggtttgttgattgcccaccagtgtcaccacccccggcaatggttggatagcgagaagaattttttaggccaaatcggcgaacacctagctgtggcgatcgtgcaatccttgctgtatagcgaagtgcaaaaacaaaaaaataactttgaaaagcgagtaattgaacggaccaaggaattgcgggataccctcatggcggcccaggcagctaacctcctcaaaagtcaatttatcaataatatttcccatgagttacgcactcctttgaccagtattatcggtctttctgctactttgttaagatggttcgaccatcccgcttccctacccccagctaagcaacaatattacctactaaatatccaagaaaacggtaaaaaactgctggatcaaattaacagtattatccaactgtcccagcttgaatccggccaaacagccctcaactgtcaatctttttccctgcataccctagcccagacagtgattcactccctgttgggggtggcaattaaacaacaaattaatttagagctagattaccaaattaatgtgggccaagaccaattctgcgctgatcaggaaagacttgaccaaattctgacccaattgctcaataatgcgctgaaatttactccggcggaaggcacagttatcctccgcatttggaaagaaagtaaccaggctatttttcaggtggaagatacgggcattggcatcaatgaacaacagttgccggtgttatttgaagcttttaaagtggcgggggacagctacactagcttctatgaaaccggtggcgtcggcctggccttaaccaagcagttggtggaactccatggcgggtatatcgaagtggaatctagccctgggcaagggacaatttttaccactgtaatcccccagcagaatttccccccaaccaccaagggacaagtgcaggataagttggatgccgccatgccctttaattccagtgtaattgtcatcgaacaggatgaggaaattgccacgctgatttgtgaattattaactgtggctaactatcaggtcatttggttaattgatactacgaatgctttgcagcaggtggaactgttacaaccgggtttgattattgtggatggggattttgtcgatgtaacggaagtgaccaggggtattaaaaagtcccggcgcatcagtaaggtcacggtttttctgctgagtgagtccctatcctcagcggagtggcaggccttatcccaaaaaggtattgacgattacttgctcaaacccctccagccagaattacttttacaacgggtgcagagtatccagcaggaacccctccgctaaattttttacgattaatgcttggactatagattaatgttttttggagtttctatcctcgaaataccgataagatattaagattgaggggaattgcaggaaaactgatgagcagatttgatagcggattacccagtgtccgccaagtacagcttttgatcaaggatcaaaccccggtggaaatcaaactactaacgggggattccctcttcggcaccattcgttggcaagataccgatggcctagggctagtcgatgactctgaacgttctaccattgtgcgcctagcggcgatcgcctacattactccccgccgttaggaattaatcatccagtcaaagcagttatagccattgggacgattaggcaatattgtcaaaccatttctttatggcccgttgataaaactgagctaatctccaatctctataaccaccatctgccacataactctgtacttttttcctccgccaatcagcctttctaccccgcactaagcccccccatgagcatccaagaaatctttaccaaagccctccaggatggatacctcacccccgctatggaagcagaagtgggacgtctttgtgaaagtggagtggatttagaccaaggggaatacgaagcgctcgatcgcctgatggcggccctgttggctggggacgtggtggccatgccccacaagaaatttattaacgtcatggaggaaatggtcctcactgaggtggtttcccaggtgtctaaatatcagaagaccacggaaaaacaacccgatattgccgacattgccgcctatgccctcaaccgcctaccaccactgtacgccacctccgaagagggcgccgaatatcaacggcaacgggcctctgaagaactggagtttcttattcaacaacaggtgaaagatggcttgggcagatattttgatcggcctcaaatcgctgaccgtaagcccctagaaccattagtgaaacaggatctgatcagtcaaatggcattgttattggaagccctagcccaggattgagcgcaaaatagcacaaattaagaactgccataggccatagtcttagccccagtctccggccataggattaatttatctgctggtgattttccctgacttaatcgtgttttccaacaggttttaccctggttggttccggttatcttcatacttttttaggggaaccaccgccgatgttgtggattaattgggcctagtgcaagatgggttttcgtcgctatcaaagtttgtttgcttggattttaaccgttgttctcaccgccactgtcaccgttgccattgcttccccggtacgggcccagtcctggatgaatttattgctccaaggattacaggtgattcaactttccaatctttctgaccagcaggaagtacggctagggcaacagattaatcagcaactttaccaacaaggagaatttcgtccccacggcgatcgccaattgcaaagttatattaacgaaattggtcaacggttagcccgggccagtagtcgccccaatatcccctacacattccaggtggtagatgaccgcagaattaacgcctttgccaccatggggggctttgtttatgtgaataccggcttgattgccgccgccgaaaatgaagcggaattagccggtgtcattgcccacgaaattgcccacattgccgaacgccatgccgtgacaagaatgagggatgtgggtctttcccaaggattaatttccgccgccggtatgcgggaaaataatattgtccagatggctatgcaactgggtttcaatctgcccatgagtagggaagatgaattaagcgccgatgccatggggctaaggaacctcgtgcgggctggttatgcccccgtaggcatggttagtttcatggaaaaattacaacggcagagtggtgccgctccagagtttcttagcagtcatcccctcacccaaaatcgcatcacttccctccagaggagtatcccgtcccaatacctctaccaaggagacggattggatccccaggcttatagccgacgcattggccgccaatcctctgcccccaccctgcgttagggtaattcctctagtaaaacagtgaaagtgacggtggaccccagtccttcccccatgctaaaaaaggccactttgccccccatggtttccaccaacttttgagaaattaccagccctaaccccgtacctccataggctttggaatgggagccatcaatttgcacaaattttttaaataatttggactgttgctcgagggaaactccaatgcccgtatctcccacgctcacttttaataatcctggtttctgtacaccttcccagagcacagtgcggggcattacttccacggccactgtaatacctccttcgtgggtaaatttgatggcgttgccgacaatattgagcatcacctgcagtaaccaacgctgattgccatacaccagcagagggccttggttgggggcagtgagattaaagctgaggcccttattttgggcctggggcagggcaaaattttctgttgcctggagcacttcactaaagtccaccacttccaattcaatgcccactcgacccgcttcaattttggccaggtcgagaatatcattaattaaattgagcaataacagggcagattggtaggcttcttcgacgaattcgtgtttttccgcctctgaatccgccatgtcgtctaaaattagccgcagaaaaccaataatgccatttaatggggtgcggagttcgtgggtggtgctggccagaaattcacttttgagccgggatgcttcttctgccgctttggtggcctgggttagttcttggtagagggtggcatgggcgatcgccgtgccggtttgctcggccaattcctgaagtagggcttctgtttctggatgccagggaccaggggccggaccaacttgttgcaaacacagatagccgttggcttggtcctggtagagggtggggaaaatccacagtccccccaaatcctccccttgaaactggacattgtagagagtgatgggttggtgttgctcgaataactgccgccaaatttcactttcctccggcacaattagagcagcgaaggcggtcactgtgggctgacagaattctgcttcaatctgcaggtggcgactttggaaatcctttgtcaacagtaggcaacggcttacccccaaggtagagccaatactatccactgcctcttgccaaatggtgggcagatggaggctattgcgtcggatccttttggcgatcgccgtcaattgggaaataaccgggggatgcagaggatccaaagaagaaaatctgagggtggattccgtcaaatcgctcggctgtcccatgtcaaaaagtggttaggtaatggagtcagcggaggaaatagcataatcaaccatggggtctatttctcccgtaggaagtcaaaaaattagtcgttccactctccccggggaggaacagggggattgcggcgtaaattgcgggttagctcatcgtcccgttgtttttcaccccgtagccactgcttcacagccatttcaattacccgatttggctcattggtcaaatgctgaatttgatccattagatccgagtccaactgaatggaaacttctattttttcagagtcccgaagcgaggcggtggtattgtcattcatggcgttagaaacgaggtcttcaccaattatagtatcccatcagtttcaggccaggcggatataatatatggaacttcaggaaaagttaccaattgggaaagtccgtccagattgaacaatttttgtcaacatcccccttcctgccacctccaagttatcaacgccagtttcataactccaccatgttagacgccgtcattattctcctctccattttcacctttgcaggggtcggcttcgccgtggtggacctgttaccggattccgtacagatgcaaatttccaacatggaggccctgcgttggctggcggcgggtttcatgtccattattggtctggcgatcgggttagtggcccaaaccacctatcgcagattggaaaatcagattcgacaaactcccattgagacaattcttaccagggcagtgggtttggtaattggtttgttgattgctaatctcatgctagctcccatctttctactgcccattccaggggaattttcctttattaagcccaccctggcagtgtttggcagtgtggtttttgcttttcttggtatcagtttggccgacacccatggccgcaccttcatgcgcctaattaaccccaacagcattgaaaccatgttgttggccgagggcaccctcaagcccatcccgcctaaagttgttgataccagttgcatcatcgacggtcgcattgagcccctattagccaccggctttattgaagggcaggtattagtgccccaatttgtcttggcagaactgcaacaattagccgatgccagcaatgaccaaaaacgggtgcgaggccggaggggattagatattctcggcagtatccaggcggattttcccgaaagcatcgttattcattccgctgactatgacgacattgccacggtggatgccaaattagtccatttagcccaagaaatcaatggcattttgctcaccaatgattacaacctcagcaaagttgccaacttgcaaaaagtccaaattctcaacgtgaacgatttggcccaggcagtgcggcccatttacctaccaggggattctctggagctaaaaatcatcaaacaaggcaaggaagcttcccagggagtaggttacctagaagatggcaccatggtggtggtggaagaagggcgcaactatgtagggtcagaattgcaggtcattgtcacttcagctttgcaaacctccgccggtcggatgatttttgccaaacccccttcagtgctggcttcttaggtcaaaattggcagtttttcagaaacggcaaaacgatttcccatcagatccgcttttccctagggagcatcctgccattctggagacaacgtaataaagtcaaaaacttctgccccggggtgacaggtgatacaggtcttacttgtgacttcctccggcataggggcccgtggatgtagagctttgaaaaagcgtgatttttccgcatagagggggactgggttatttggaggcagtggtcgagaaaacgtactcacataatcccaaatcagcaattgggtcaggcgattaatgttaggaatgctgaccccataatggacatccggacgacgcaaaatctcctgccaggaatccaggggtaagacttccggtggcagggcaatgtgacagccagtacaggtttcgatgtaggactcgaaaccaggaaggtaacgggtag

SEQ ID NO: 5 - DNA, 1461 bp, Artificial Sequence
  Description of Artificial Sequence Synthetic polynucleotide
  A modified sequence encoding kanamycin nucleotidyltransferase derived from Bacillus stearothermopilus.
gatctgttaacagctgattgattgagcaagctttatgcttgtaaaccgttttgtgaaaaaatttttaaaataaaaaaggggacctctagggtccccaattaattagtaatataatctattaaaggtcattcaaaaggtcatccagacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaaatctggatgaccttttgaatgacctttaatagattatattactaattaattggggaccctagaggtccccttttttattttaaaaattttttcacaaaacggtttacaagcataaagcttgctcaatcaatcagctgttaaca

SEQ ID NO: 6 - DNA, 17567 bp, Homo sapiens
tgcacctgctctgtgattatgactatcccacagtctcctggttgtctacccatggacctagaggtactttgaaagttttggatatctgggctctgactgtgcaataatgggcaaccccaaagtcaaggcacatggcaagaaggtgctgatctccttcggaaaagctgttatgctcacggatgacctcaaaggcacctttgctacactgagtgacctgcactgtaacaagctgcacgtggaccctgagaacttcctggtgagtagtaagtacactcacgctttcttctttacccttagatatttgcactatgggtacttttgaaagcagaggtggctttctcttgtgttatgagtcagctatgggatatgatatttcagcagtgggattttgagagttatgttgctgtaaataacataactaaaatttggtagagcaaggactatgaataatggaaggccacttaccatttgatagctctgaaaaacacatcttataaaaaattctggccaaaatcaaactgagtgttttggatgagggaacagaagttgagatagagaaaataacatctttcctttggtcagcgaaattttctataaaaattaatagtcacttttctgcatagtcctggaggttagaaaaagatcaactgaacaaagtagtgggaagctgttaaaagaggattgtttccctccgaatgatgatggtatacttttgtacgcatggtacaggattctttgttatgagtgtttgggaaaattgtatgtatgtatgtatgtatgtgatgactggggacttatcctatccattactgttccttgaagtactattatcctactttttaaaaggacgaagtctctaaaaaaaaaatgaaacaatcacaatatgttggggtagtgagttggcatagcaagtaagagaaggataggacacaatgggaggtgcagggctgccagtcatattgaagctgatatctagcccataatggtgagagttgctcaaactctggtcaaaaaggatgtaagtgttatatctatttactgcaagtccagcttgaggccttctattcactatgtaccattttcttttttatcttcactccctccccagctcttaggcaacgtgatattgattgttttggcaacccacttcagcgaggattttaccctacagatacaggcttcttggcagtaactaacaaatgctgtggttaatgctgtagcccacaagaccactgagttccctgtccactatgtttgtacctatgtcccaaaatctcatctcctttagatgggggaggttggggagaagagcagtatcctgcctgctgattcagttcctgcatgataaaaatagaataaagaaatatgctctctaagaaatatcattgtactctttttctgtctttatattttaccctgattcagccaaaaggacgcactatttctgatggaaatgagaatgttggagaatgggagtttaaggacagagaagatactttcttgcaatcctgcaagaaaagagagaactcgtgggtggatttagtggggtagttactcctaggaaggggaaatcgtctctagaataagacaatgtttttacagaaagggaggtcaatggaggtactctttggaggtgtaagaggattgttggtagtgtgtagaggtatgttaggactcaaattagaagttctgtataggctattatttgtatgaaactcaggatatagctcatttggtgactgcagttcacttctacttattttaaacaacatattttttatgatttataatgaagtggggatggggcttcctagagaccaatcaagggccaaaccttgaactttctcttaacgtcttcaatggtattaatagagaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtacaagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctgagatttaggaaacagcacgttaagtcaagttgatagaggagaatatggacatttaaaagaggcaggatgatataaaattagggaaactggatgcagagaccagatgaagtaagaaaaatagctatcgttttgagcaaaaatcactgaagtttcttgcatatgagagtgacataataaatagggaaacgtagaaaattgattcacatgtatatatatatatagaactgattagacaaagtctaacttgggtatagtcagaggagcttgctgtaattatattgaggtgatggataaagaactgaagttgatggaaacaatgaagttaagaaaaaaaatcgagtaagagaccattgtggcagtgattgcacagaactggaaaacattgtgaaacagagagtcagagatgacagctaaaatccctgtctgtgaatgaaaagaaggaaatttattgacagaacagcaaatgcctacaagccccctgtttggatctggcaatgaacgtagccattctgtggcaatcacttcaaactcctgtacccaagacccttaggaagtatgtagcaccctcaaacctaaaacctcaaagaaagaggttttagaagatataataccctttcttctccagtttcattaatcccaaaacctctttctcaaagtatttcctctatgtgtccaccccaaagagctcacctcaccatatctcttgagtgggagcacatagataggcggtgctaccatctaacagcttctgaaattcctttgtcatatttttgagtccccactaataacccacaaagcagaataaataccagttgctcatgtacaataatcactcaactgctgtcttgtagcatacattaattaagcacattctttgaataattactgtgtccaaacaatcacactttaaaatctcacacttgtgctatcccttgcccttctgaatgtcactctgtattttaaatgaagagatgagggttgaatttcctgtgttacttattgttcatttctcgatgaggagttttcacattcacctttactggaaaacacataagtacacatcttacaggaaaaatataccaaactgacatgtagcatgaatgcttgtgcatgtagtcatataaaatcttgtagcaatgtaaacattctctgatatacacatacagatgtgtctatatgtctacacaatttcttatgctccatgaacaaacattccatgcacacataagaacacacactgttacagatgcatacttgagtgcattgacaaaattaccccagtcaatctagagaatttggatttctgcatttgactctgttagctttgtacatgctgttcatttactctgggtgatgtctttccctcattttgccttgtctatcttgtactcatactttaagtcctaacttatatgttatctcaactaagaagctatttttttttaattttaactgggcttaaagccctgtctataaactctgctacaattatgggctctttcttataatatttagtgtttttcctactaatgtacttaatctgctcattgtatattcctaccactaaattttaacctcttttatggtagagacattgtcttgtaaactcttatttccctagtatttggagatgaaaaaaaagattaaattatccaaaattagatctctcttttctacattatgagtattacactatccatagggaagtttgtttgagacctaaactgaggaacctttggttctaaaatgactatgtgatatcttagtatttataggtcatgaggttccttcctctgcctctgctatagtttgattagtcagcaagcatgtgtcatgcatttattcacatcagaatttcatacactaataagacatagtatcagaagtcagtttattagttatatcagttagggtccatcaaggaaaggacaaaccattatcagttactcaacctagaattaaatacagctcttaatagttaattatccttgtattggaagagctaaaatatcaaataaaggacagtgcagaaatctagatgttagtaacatcagaaaacctcttccgccattaggcctagaagggcagaaggagaaaatgtttataccaccagagtccagaaccagagcccataaccagaggtccactggattcagtgagctagtgggtgctccttggagagagccagaactgtctaatgggggcatcaaagtatcagccataaaaaaccataaaaaagactgtctgctgtaggagatccgttcagagagagagagagaccagaaataatcttgcttatgctttccctcagccagtgtttaccattgcagaatgtacatgcgactgaaagggtgaggaaacctgggaaatgtcagttcctcaaatacagagaacactgagggaaggatgagaaataaatgtgaaagcagacatgaatggtaattgacagaaggaaactaggatgtgtccagtaaatgaataattacagtgtgcagtgattattgcaatgattaatgtattgataagataatatgaaaacacagaattcaaacagcagtgaactgagattagaattgtggagagcactggcatttaagaatgtcacacttagaatgtgtctctaggcattgttctgtgcatatatcatctcaatattcattatctgaaaattatgaattaggtacaaagctcaaataatttattttttcaggttagcaagaactttttttttttttttttctgagatggagcattgctatggttgcccaggctggagtgcaatggcatgatccaggctcactgcaacatctgcctcccaggttcaagcgattctcctgcctcagcctcccaagtagctggcattacaggcatgtgccaccaccatgcctggctaattttctatttttagtagatagggggtttcaccatgttggtcaggctgatctcgaactcctaacatcaggtgatccaccctcctcggcctctgaatgtactgggatcacaggcgtgagccaccacacccagccaagaatgtgaattttgtagaaggatataacccatatttctctgaccctagagtccttagtatacctcccataccatgtggctcatcctccttacatacatttcccatctttcaccctaccttttcctttttgtttcagcttttcactgtgtgtcaaaatctagaaccttatctcctacctgctctgaaaccaacagcaagttgacttccattctaacccacattggcattacactaattaaaatcgatactgagttctaaaatcatctgggattttggggactatgtcttacttcatacttccttgagatttcacattaaatgttggtgttcattaaaggtccttcatttaactttgtattcatcacactcttggattcacagttatatctaaactcttatatatagcctgtataatcccaattcccaagtctgatttctaacctctgacctccaacctcagtgccaaacccatatatcaaacaatgtactgggcttatttatatagatgtcctataggcacctcagactcagcatgggtatttcacttgttatactaaaactgtttctcttccagtgttttccattttagtcattagatagctacttgcccattcaccaaggtcacagattaaaatcatttccctacctctaatcaacagttcaattctgcttcaatttgtccctatctattaatcaccactcttactgcccagtcaggtcctcattgtttcctgaacaagagtagatgctattctttccactttaagaccttatcctggctggatgcggtggctcaggcttgtaaacccagcactttgggaggccgaggcaggcagatcacttgaggtcaggagttcaagaccagcctgaccaacatggtgaaaccccatctctactaaaaatacaaaatcagccgggcgtgtggtgcatgcctgcagtcccagctattcaggtggctgaggcaggagaattgcttgaacccaggaggcggaggttgcggtgagcctagattgcaccattgcactctagcttgggcaatagggatgaaactccatctcagaagagaaaagaaaaaaagaccttattctgttacacaaatcctctcaatgcaatccatatagaataaacatgtaaccagatctcccaatgtgtaaaatcatttcaggtagaacagaattaaagtgaaaagccaagtctttggaattaacagacaaagttcaaataacagtcctcatggccttaagaatttacctaacattttttttagaatcaattttcttatatatgaattggaaacataattcctccctcacaaacacattctaagattttaaggagatattgatgaagtacatcatctgtcatttttaacagttagtggtagtgattcacacagcacattatgatctgttcttgtatgttctgttccattctgtattcttgacctggttgtattctttctgagctccagatccacatatctaagtacatctttttgcattttacaagagtgcatacaatacaatgtatccaagactgtatttctgattttatcgtaccactaaactcacaaatgtggccctattcttgtgttcacgactgacatcaccgtcatggtccaagtctgataatagaaatggcattgtcactttcttccctactgcaacagaagcccagctatttgtctcccattttctctacttctaaaatacatttcttcactaagtgagaataatcttttaaagacacaaatcaaaccatgccaccacctttcttgaattattcaatatctttcgttggcttccaggttacagaaaaataacttgtaacaaagtttaaaggtcattcatggctcctctctaccctattttataacatttccccttgtgatcagaatctcaggcacatcatccatctttctatatacaaataaagtcatatagtttgaactcacctctggttacttttaatcaaccaaatgctgtaaaatgcatttgtatcgctacgtgttaagcagtagttgattcttttcatttcttgttaatattctattctttgactataccgtaatttatcaattctactgttggtaagcatttaagtggctaccggtttgaggtttttatgattattgctgtcataagcatttctatacatgtctttggatacacacatgcatgtgtttctgaatatctaaaaatgtaattgctaggtaatagacttatcaagcatccagcatttgtggatactattaaaggttttccaaaggggttatactattgtacagtgtcaccaacagagtttgagtttctattgatccatatcaccaccaaaatttgaactgtcagtcttatctcttctcttgtctcttttttcctcttttttttccttcccttcccctctcttcgtttcttttctctcctcttctcttctttcctctcttcccttccctttctctttctcttccctatcccttctcctctcctctcccctccttttttctcctctcctctccattatttatttttccttcttctcctccatcccttccatcctctctcttcccctcttccttccttcctttctccatttcttcctcctctttccctcaatccttccttttggatatgctcatgggtgtgtatttgtctgccattgtggcattatttgaattcagaaaagagtgaaaaactactgggatcttcattctgggtctaattccacatttttttttaagaacacactctgtaaaaatgttctgtactagcatattcccaggaacttcgttaaatttaatctggctgaatatggtaaatctactttgcactttgcattctttctttagtcataccataattttaaacattcaaaatatttgtatataatatttgattttatctgtcattaaaatgttaaccttaaaattcatgtttccagaacctatttcaataactggtaaataaacactattcattttttaaatattcttttaatggatatttatttcaatataataaaaaattagagttttattataggaagaatttaccaaaagaaggaggaagcaagcaagtttaaactgcagcaatagttgtccattccaacctctcaaaattcccttggagacaaaatctctagaggcaaagaagaactttatattgagtcaacttgttaaaacatctgcttttagataagttttcttagtataaagtgacagaaacaaataagttaaactctaagatacattccactatattagcctaaaacacttctgcaaaaatgaaactaggaggatatttttagaaacaactgctgaaagagatgcggtggggagatatgcagaggagaacagggtttctgagtcaagacacacatgacagaacagccaatctcagggcaagttaagggaatagtggaatgaaggttcatttttcattctcacaaactaatgaaaccctgcttatcttaaaccaacctgctcactggagcagggaggacaggaccagcataaaaggcagggcagagtcgactgttgcttacactttcttctgacataacagtgttcactagcaacctcaaacagacaccatggtgcatctgactcctgaggagaagactgctgtcaatgccctgtggggcaaagtgaacgtggatgcagttggtggtgaggccctgggcaggttggtatcaaggttataagagaggctcaaggaggcaaatggaaactgggcatgtgtagacagagaagactcttgggtttctgataggcactgactctctgtcccttgggctgttttcctaccctcagattactggtggtctacccttggacccagaggttctttgagtcctttggggatctgtcctctcctgatgctgttatgggcaaccctaaggtgaaggctcatggcaagaaggtgctaggtgcctttagtgatggcctggctcacctggacaacctcaagggcactttttctcagctgagtgagctgcactgtgacaagctgcacgtggatcctgagaacttcagggtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctcttgggcaatgtgctggtgtgtgtgctggcccgcaactttggcaaggaattcaccccacaaatgcaggctgcctatcagaaggtggtggctggtgtggctaatgccctggctcacaagtaccattgagatcctggactgtttcctgataaccataagaagaccctatttccctagattctattttctgaacttgggaacacaatgcctacttcaagggtatggcttctgcctaataaagaatgttcagctcaacttcctgattaatttcacttatttcatttttttgtccaggtgtgtaagaaggttcctgaggctctacagatagggagcacttgtttattttacaaagagtacatgggaaaagagaaaagcaagggaaccgtacaaggcattaatgggtgacacttctacctccaaagagcagaaattatcaagaactcttgatacaaagataatactggcactgcagaggttctagggaagacctcaaccctaagacatagcctcaagggtaatagctacgattaaactccaacaattactgagaaaataatgtgctcaattaaaggcataatgattactcaagacaatgttatgttgtctttcttcctccttcctttgcctgcacattgtagcccataatactataccccatcaagtgttcctgctccaagaaatagcttcctcctcttacttgccccagaacatctctgtaaagaatttcctcttatcttcccatatttcagtcaagattcattgctcacgtattacttgtgacctctcttgaccccagccacaataaacttctctatactacccaaaaaatctttccaaaccctccccgacaccatatttttatatttttcttatttatttcatgcacacacacacactccgtgctttataagcaattctgcctattctctaccttcttacaatgcctactgtgcctcatattaaattcatcaatgggcagaaagaaaatatttattcaagaaaacagtgaatgaatgaacgaatgagtaaatgagtaaatgaaggaatgattattccttgctttagaacttctggaattagaggacaatattaataataccatcgcacagtgtttctttgttgttaatgctacaacatacaaagaggaagcatgcagtaaacaaccgaacagttatttcctttctgatcataggagtaatatttttttccttgagcacatttttgccataggtaaaattagaaggatttttagaactttctcagttgtatacatttttaaaaatctgtattatatgcatgttgattaattttaaacttacttgaatacctaaacagaatctgttgtttccttgtgtttgaaagtgctttcacagtaactctgtctgtactgccagaatatactgacaatgtgttatagttaactgttttgatcacaacattttgaattgactggcagcagaagctctttttatatccatgtgttttccttaagtcattatacatagtaggcatgagactctttatactgaataagatatttaggaaccactggtttacatatcagaagcagagctactcagggcattttggggaagatcactttcacattcctgagcatagggaagttctcataagagtaagatattaaaaggagatacttgtgtggtattcgaaagacagtaagagagattgtagaccttatgatcttgatagggaaaacaaactacattcctttctccaaaagtcaaaaaaaaagagcaaatatagcttactataccttctattcctacaccattagaagtagtcagtgagtctaggcaagatgttggccctaaaaatccaaataccagagaattcatgagaacatcacctggatgggacatgtgccgagcaacacaattactatatgctaggcattgctatcttcatattgaagatgaggaggtcaagagatgaaaaaagacttggcaccttgttgttatattaaaattatttgttagagtagagcttttgtaagagtctaggagtgtgggagctaaatgatgatacacatggacacaaagaatagatcaacagacacccaggcctacttgagggttgagggtgggaagagggagacgatgaaaaagaacctattgggtattaagttcatcactgagtgatgaaataatctgtacatcaagacccagtgatatgcaatttacctatataacttgtacatgtacccccaaatttaaaataaagttaaaacaaagtataggaatggaattaattcctcaagatttggctttaattttatttgataatttatcaaatggttgtttttcttttctcactatggcgttgctttataaactatgttcagtatgtctgaatgaaagggtgtgtgtgtgtgtgaaagagagggagagaggaagggaagagaggacgtaataatgtgaatttgagttcatgaaaatttttcaataaaataatttaatgtcaggagaattaagcctaatagtctcctaaatcatccatctcttgagcttcagagcagtcctctgaattaatgcctacatgtttgtaaagggtgttcagactgaagccaagattctacctctaaagagatgcaatctcaaatttatctgaagactgtacctctgctctccataaattgacaccatggcccacttaatgaggttaaaaaaaagctaattctgaatgaaaatctgagcccagtggaggaaatattaatgaacaaggtgcagactgaaatataaattttctgtaataattatgcatatactttagcaaagttctgtctatgttgactttattgcttttggtaagaaatacaactttttaaagtgaactaaactatcctatttccaaactattttgtgtgtgtgcggtttgtttctatgggttctggttttcttggagcatttttatttcattttaattaattaattctgagagctgctgagttgtgtttactgagagattgtgtatctgcgagagaagtctgtagcaagtagctagactgtgcttgacctaggaacatatacagtagattgctaaaatgtctcacttggggaattttagactaaacagtagagcatgtataaaaatactctagtcaagtgctgcttttgaaacaaatgataaaaccacactcccatagatgagtgtcatgattttcatggaggaagttaatattcatcctctaagtatacccagactagggccattctgatataaaacattaggacttaagaaagattaatagactggagtaaaggaaatggacctctgtctctctcgctgtctcttttttgaggacttgtgtgtgtgtgtgtgtgtgtgtgtgtgtgtgttgtggtcagtggggctggaataaaagtagaatagacctgcacctgctgtggcatccattcacagagtagaagcaagctcacaatagtgaagatgtcagtaagcttgaatagtttttcaggaactttgaatgctgatttagatttgaaactgaggctctgaccataaccaaatttgcactatttattgcttcttgaaacttatttgcctggtatgcctgggcttttgatggtcttagtatagcttgcagccttgtccctgcagggtattatgggtaatagaaagaaaagtctgcgttacactctagtcacactaagtaactaccattggaaaagcaacccctgccttgaagccaggatgatggtatctgcagcagttgccaacacaagagaaggatccatagttcatcatttaaaaaagaaaacaaaatagaaaaaggaaaactatttctgagcataagaagttgtagggtaagtctttaagaaggtgacaatttctgccaatcaggatttcaaagctcttgctttgacaattttggtctttcagaatactataaatataacctatattataatttcataaagtctgtgcattttctttgacccaggatatttgcaaaagacatattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgtatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgactgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaagctcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgt

SEQ ID NO: 7 - DNA, 22 bp, Artificial Sequence
  Description of Artificial Sequence Synthetic primer
ccagtcacgacgttgtaaaacg

SEQ ID NO: 8 - DNA, 22 bp, Artificial Sequence
  Description of Artificial Sequence Synthetic primer
cgccaagctatttaggtgagac

SEQ ID NO: 9 - DNA, 43 bp, Artificial Sequence
  Description of Artificial Sequence Synthetic primer
gatatcgtcgaccactgcacctgctctgtgattatgactatcc

SEQ ID NO: 10 - DNA, 42 bp, Artificial Sequence
  Description of Artificial Sequence Synthetic primer
tctagaacgcgtcacacatgattagcaaaagggcctagcttg

SEQ ID NO: 11 - DNA, 35 bp, Artificial Sequence
  Description of Artificial Sequence Synthetic primer
tgcacctgctctgtgattatgactatcccacagtc

SEQ ID NO: 12 - DNA, 35 bp, Artificial Sequence
  Description of Artificial Sequence Synthetic primer
agtggccttccattattcatagtccttgctctacc

SEQ ID NO: 13 - DNA, 24 bp, Artificial Sequence
  Description of Artificial Sequence Synthetic primer
gaaactggatgcagagaccagatg

SEQ ID NO: 14 - DNA, 24 bp, Artificial Sequence
  Description of Artificial Sequence Synthetic primer
aactatagcagaggcagaggaagg

SEQ ID NO: 15 - DNA, 24 bp, Artificial Sequence
  Description of Artificial Sequence Synthetic primer
accacactcccatagatgagtgtc

SEQ ID NO: 16 - DNA, 24 bp, Artificial Sequence
  Description of Artificial Sequence Synthetic primer
ctctatagcttcccaacgtgatcg

SEQ ID NO: 17 - DNA, 35 bp, Artificial Sequence
  Description of Artificial Sequence Synthetic primer
cagtctgcctagtacattactatttggaatatatg

SEQ ID NO: 18 - DNA, 35 bp, Artificial Sequence
  Description of Artificial Sequence Synthetic primer
acatgattagcaaaagggcctagcttggactcaga

SEQ ID NO: 19 - DNA, 61 bp, Artificial Sequence
  Description of Artificial Sequence Synthetic oligonucleotide
cggggatcccacgtacaacgacacctagaccacgtgttcctaggctgtttcctggtgggat

SEQ ID NO: 20 - DNA, 38 bp, Unknown
  Description of Unknown cyanobacterium gene transfer site sequence
ggatccgcggccgcgatatcgtcgaccacgtgacgcgt

(19) United States
(12) Patent Application Publication
(10) Pub. No.: US 2022/0411744 A1
(43) Pub. Date: Dec. 29, 2022

(54) CYANOBACTERIUM, METHOD FOR PRODUCING CYANOBACTERIUM, AND GENE TRANSFER VECTOR

(71) Applicant: TOYOTA BOSHOKU KABUSHIKI KAISHA, Aichi (JP)

(72) Inventors: Masaro AKAI, Aichi (JP); Naoko KAMIYA, Aichi (JP)

(73) Assignee: TOYOTA BOSHOKU KABUSHIKI KAISHA, Aichi (JP)

(21) Appl. No.: 17/843,383

(22) Filed: Jun. 17, 2022

(30) Foreign Application Priority Data
Jun. 18, 2021 (JP) 2021-101410

(51) Int. Cl. C12N 1/12; C12N 15/90; C12N 15/74

(52) U.S. Cl. CPC C12N 1/125; C12N 15/902; C12N 15/74; C12N 2800/101; C12R 2001/01

(57) ABSTRACT

A cyanobacterium transformed using a gene transfer vector. The gene transfer vector includes: a first homologous recombination region homologous to a 5′ side of a target DNA of the cyanobacterium; a second homologous recombination region homologous to a 3′ side of the target DNA; and a DNA fragment introduced into a portion sandwiched between the first homologous recombination region and the second homologous recombination region. A total length of the first homologous recombination region and the second homologous recombination region is 5 kbp or more. The cyanobacterium has a DNA fragment transferred between the 5′ side of the target DNA and the 3′ side of the target DNA.

### CROSS-REFERENCE TO RELATED APPLICATION

This application is based on and claims the priority of Japanese Patent Application No. 2021-101410 filed on Jun. 18, 2021, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Jun. 3, 2022, is named P65963_SL.txt and is 127,979 bytes in size.

### BACKGROUND OF THE INVENTION

### (1) Field of the Invention

The present disclosure relates to a cyanobacterium, a method for producing a cyanobacterium, and a gene transfer vector.

### (2) Description of Related Art

WO 2014/126225 describes that a relatively long DNA fragment can be introduced by using an electroporation method as a method for introducing a targeting vector into ES cells.

JP 2004-147538 A describes a transformation method using an agrobacterium as a genetic recombination method for plants. It describes that, in this technique, that a long DNA fragment can be introduced, and that the introduced gene is also stably retained.

### SUMMARY OF THE INVENTION

Cyanobacteria have advantageous properties as media (hosts) for the production of useful substances, such as fast growth and high photosynthesis capacity. Therefore, cyanobacteria have been improved by genetic recombination. However, conventional techniques have a problem that the length of DNA which can be introduced into a genomic DNA of cyanobacterium is limited, making it difficult to obtain useful cyanobacterium.

The present disclosure has been made in view of the above circumstances, and an object thereof is to provide a useful cyanobacterium. Another object of the present invention is to provide a method for producing a useful cyanobacterium and a gene transfer vector used for obtaining a useful cyanobacterium.

The present disclosure can be realized as the following forms.

[1] A cyanobacterium transformed using a gene transfer vector,
the gene transfer vector including:
   a first homologous recombination region homologous to a 5′ side of a target DNA of a cyanobacterium; a second homologous recombination region homologous to a 3′ side of the target DNA; and a DNA fragment introduced into a portion sandwiched between the first homologous recombination region and the second homologous recombination region,
wherein a total length of the first homologous recombination region and the second homologous recombination region is 5 kbp or more, and
wherein the DNA fragment is transferred between the 5′ side of the target DNA and the 3′ side of the target DNA.

According to the present disclosure, a useful cyanobacterium can be provided. Also, it is possible to provide a method for producing a useful cyanobacterium and a gene transfer vector used for obtaining a useful cyanobacterium.

### BRIEF DESCRIPTION OF THE DRAWINGS

FIG. 1 is a diagram conceptually showing a gene transfer vector;

FIG. 2 is a diagram showing a first homologous recombination region and a second homologous recombination region in an example of the gene transfer vector;

FIG. 3 is a diagram showing a first homologous recombination region and a second homologous recombination region in another example of the gene transfer vector;

FIG. 4 is a diagram showing a fosmid vector used for construction of the gene transfer vector. FIG. 4 discloses SEQ ID NO: 19;

FIG. 5 is a diagram showing a multi-cloning site. FIG. 5 discloses SEQ ID NO: 20;

FIG. 6 is a diagram showing the vicinity of a gene transfer site in a genomic DNA of a transformed cyanobacterium.

FIG. 7 is a photograph showing a result of confirming a band of DNA derived from an introduced DNA fragment by agarose electrophoresis; and

FIG. 8 is a photograph showing a result of analyzing the genomic DNA of the transformed cyanobacterium by southern blotting.

### DETAILED DESCRIPTION OF PREFERRED EMBODIMENTS

Here, desirable examples of the present disclosure are presented.

[2] The cyanobacterium, wherein a transfer site of the DNA fragment in the target DNA is a site where 3′ side untranslated regions of adjacent endogenous genes face each other.

[3] The cyanobacterium, wherein the transfer site of the DNA fragment in the target DNA is a site where 3′ side untranslated regions of an slr 1716 gene and an sll 1609 gene in a cyanobacterium Synechocystis sp. PCC 6803 strain face each other.

[4] The cyanobacterium, wherein the transfer site of the DNA fragment in the target DNA is a site where 3′ side untranslated regions of an slr 1966 gene and an sll 1893 gene in a cyanobacterium Synechocystis sp. PCC 6803 strain face each other.

[5] The cyanobacterium, wherein the DNA fragment is 5 kbp or more.

[6] A method for producing a cyanobacterium including transforming the cyanobacterium using a gene transfer vector,
the gene transfer vector including:
   a first homologous recombination region homologous to a 5′ side of a target DNA of a cyanobacterium; a second homologous recombination region homologous to a 3′ side of the target DNA; and a DNA fragment introduced into a portion sandwiched between the first homologous recombination region and the second homologous recombination region,
wherein a total length of the first homologous recombination region and the second homologous recombination region is 5 kbp or more, and
wherein, in the transforming, the DNA fragment is transferred between the 5′ side of the target DNA and the 3′ side of the target DNA.

[7] The method for producing a cyanobacterium, wherein a transfer site of the DNA fragment in the target DNA is a site where 3′ side untranslated regions of adjacent endogenous genes face each other.

[8] The method for producing a cyanobacterium, wherein the transfer site of the DNA fragment in the target DNA is a site where 3′ side untranslated regions of an slr 1716 gene and an sll 1609 gene in a cyanobacterium Synechocystis sp. PCC 6803 strain face each other.

[9] The method for producing a cyanobacterium, wherein the transfer site of the DNA fragment in the target DNA is a site where 3′ side untranslated regions of an slr 1966 gene and an sll 1893 gene in a cyanobacterium Synechocystis sp. PCC 6803 strain face each other.

[10] The method for producing a cyanobacterium, wherein the DNA fragment is 5 kbp or more.

[11] A gene transfer vector used for a cyanobacterium, including:
a first homologous recombination region homologous to a 5′ side of a target DNA of the cyanobacterium; and a second homologous recombination region homologous to a 3′ side of the target DNA,
wherein a total length of the first homologous recombination region and the second homologous recombination region is 5 kbp or more.

[12] The gene transfer vector, which has a selection marker region in a portion sandwiched between the first homologous recombination region and the second homologous recombination region.

[13] The gene transfer vector, which has a multi-cloning site for introducing a DNA fragment in a portion sandwiched between the first homologous recombination region and the second homologous recombination region.

[14] The gene transfer vector, which is a fosmid vector.

Hereinafter, the present disclosure will be described in detail. In addition, in the present specification, a phrase about a numerical range using the word “to” includes a lower limit value and an upper limit value unless otherwise specified. For example, the phrase “10 to 20” includes both the lower limit “10” and the upper limit “20”. That is, the phrase “10 to 20” has the same meaning as “10 or more and 20 or less”.

### 1. Cyanobacterium

A cyanobacterium according to the present embodiment is a cyanobacterium transformed using a gene transfer vector 10. The gene transfer vector 10 includes: a first homologous recombination region 11 homologous to a 5′ side of a target DNA of the cyanobacterium; a second homologous recombination region 12 homologous to a 3′ side of the target DNA; and a DNA fragment 25 introduced into a portion sandwiched between the first homologous recombination region 11 and the second homologous recombination region 12. A total length of the first homologous recombination region 11 and the second homologous recombination region 12 (hereinafter, also referred to as “length of the homologous recombination regions 11 and 12”) is 5 kbp or more. The cyanobacterium has a DNA fragment 25 transferred between the 5′ side of the target DNA and the 3′ side of the target DNA.

The genus and strain of the cyanobacterium to which the technology of the present disclosure is applied are not particularly limited. The cyanobacterium is preferably a cyanobacterium Synechocystis sp. PCC 6803 strain from the viewpoint that its genomic DNA is well analyzed. The names and the like of genes in the present disclosure are estimated gene numbers assigned in the genome project achieved by Kazusa DNA Research Institute. In addition, in the present disclosure, base sequences and the like are described based on the genome data on cyanobacteria published in CyanoBase and KEGG (Kyoto Encyclopedia of Genes and Genomes).

### (1) Gene Transfer Vector 10

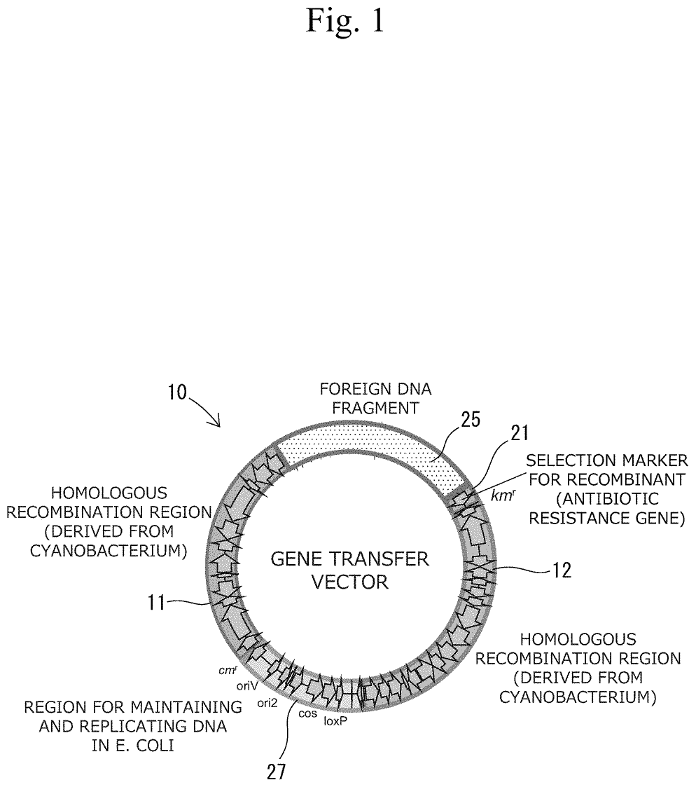

FIG. 1 is a diagram conceptually showing a gene transfer vector 10 according to the present embodiment. The gene transfer vector 10 has the DNA fragment 25 introduced into a portion sandwiched between the first homologous recombination region 11 and the second homologous recombination region 12. In the present disclosure, the “gene transfer vector” may be in a state where the DNA fragment 25 is introduced as shown in FIG. 1, or may be in a state before the DNA fragment 25 is introduced.

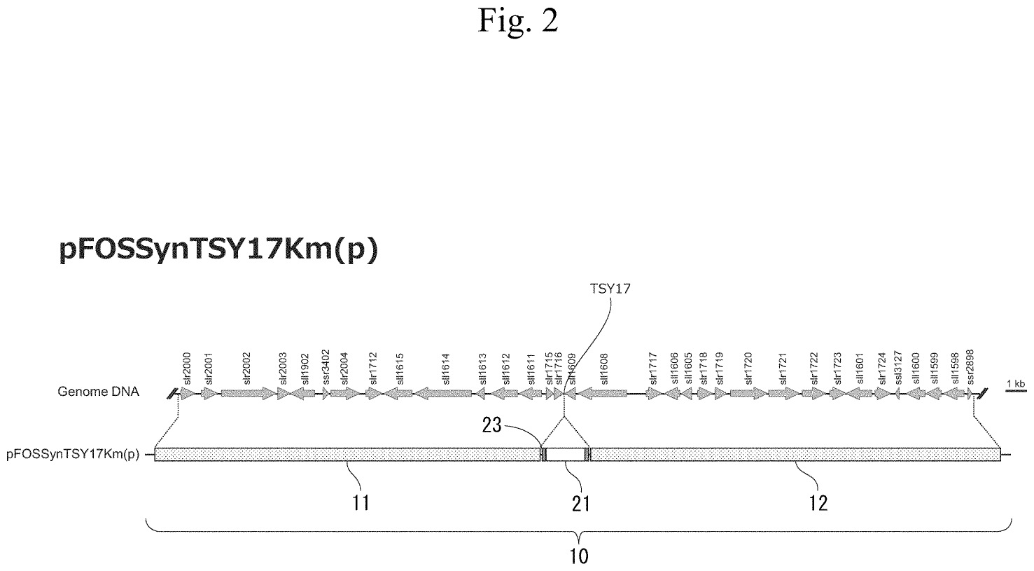

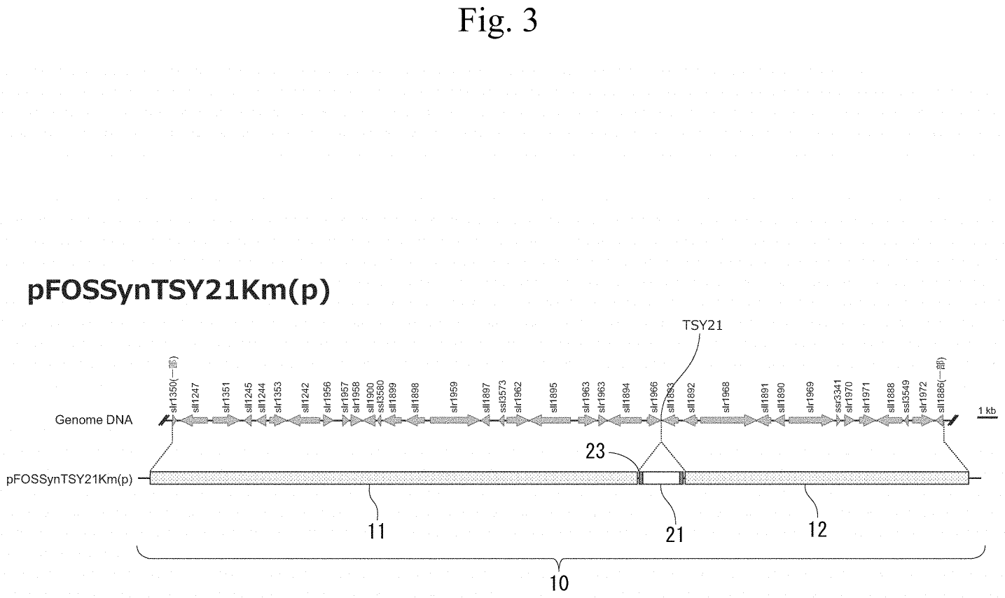

The gene transfer vector 10 preferably has a selection marker region 21 in a portion sandwiched between the first homologous recombination region 11 and the second homologous recombination region 12, as shown in FIGS. 2 and 3. The gene transfer vector 10 preferably has a multi-cloning site 23 for introducing the DNA fragment 25 in a portion sandwiched between the first homologous recombination region 11 and the second homologous recombination region 12. Hereinafter, the respective regions will be described in order.

### (1-1) First Homologous Recombination Region 11 and Second Homologous Recombination Region 12

The first homologous recombination region 11 and the second homologous recombination region 12 mean a pair of DNA regions having homology to the target DNA in the genomic DNA of cyanobacterium. When the first homologous recombination region 11 and the second homologous recombination region 12 cross over with respective DNA regions having homology, the DNA fragment 25 between the first homologous recombination region 11 and the second homologous recombination region 12 can be transferred into the genomic DNA. Base sequences of the homologous recombination regions 11 and 12 are not particularly limited, but have sequence identity high enough to be able to undergo homologous recombination with the target DNA. Identities of the base sequences with the target DNA in the homologous recombination regions 11 and 12 can be, for example, 60% or more, preferably 80% or more, more preferably 90% or more, still more preferably 95% or more, and particularly preferably 99% or more.

The length of the homologous recombination regions 11 and 12 is 5 kbp or more, preferably 10 kbp or more, more preferably 20 kbp or more, still more preferably 25 kbp or more, and still more preferably 30 kbp or more. When the length of the homologous recombination regions 11 and 12 is a lower limit value or more, a relatively long (for example, 5 kbp or more) DNA fragment 25 can be efficiently introduced into the genomic DNA of cyanobacterium. The length of the homologous recombination regions 11 and 12 is 60 kbp or less, preferably 50 kbp or less, and more preferably 40 kbp or less. When the length of the homologous recombination regions 11 and 12 is an upper limit value or less, the number of restriction enzyme sites can be reduced, which is advantageous for gene manipulation. That is, a restriction enzyme site (cloning site) for introducing a DNA fragment is easily used. From these viewpoints, the length of the homologous recombination regions 11 and 12 is preferably 5 kbp or more and 60 kbp or less, more preferably 10 kbp or more and 50 kbp or less, and still more preferably 25 kbp or more and 40 kbp or less.

The lengths of the first homologous recombination region 11 and the second homologous recombination region 12 may be the same, or may be different to the extent that homologous recombination can be performed. A difference in length between the first homologous recombination region 11 and the second homologous recombination region 12 can be 12 kbp or less, 8 kbp or less, 5 kbp or less, 3 kbp or less, or 2 kbp or less, from the viewpoint of recombination efficiency.

The length of the first homologous recombination region 11 is preferably 2.5 kbp or more and 30 kbp or less, more preferably 5 kbp or more and 25 kbp or less, and still more preferably 12 kbp or more and 22 kbp or less.

The length of the second homologous recombination region 12 is preferably 2.5 kbp or more and 30 kbp or less, more preferably 5 kbp or more and 25 kbp or less, and still more preferably 12 kbp or more and 22 kbp or less.

A transfer site of the DNA fragment 25 in the target DNA is preferably a site where 3′ side untranslated regions of adjacent endogenous genes face each other. When the transfer site of the DNA fragment 25 is located in the untranslated region, the DNA fragment 25 can be transferred without damaging a gene on the genomic DNA of cyanobacterium. In addition, it is preferable that the transfer site of the DNA fragment 25 be a site where the 3′ side untranslated regions of adjacent endogenous gene face each other, since transcriptional regulatory sites or the like existing in 5′ side untranslated regions of the endogenous genes are less likely to be damaged.

FIGS. 2 and 3 show examples of the transfer site of the DNA fragment in the cyanobacterium Synechocystis sp. PCC 6803 strain.

In the gene transfer vector 10 shown in FIG. 2, a transfer site TSY17 of the DNA fragment 25 in the target DNA is a site where 3′ side untranslated regions of an slr 1716 gene and an sll 1609 gene in the cyanobacterium Synechocystis sp. PCC 6803 strain face each other.

In the gene transfer vector 10 shown in FIG. 3, a transfer site TSY21 of the DNA fragment 25 in the target DNA is a site where 3′ side untranslated regions of an slr 1966 gene and an sll 1893 gene in the cyanobacterium Synechocystis sp. PCC 6803 strain face each other.

The site at which the 3′ side untranslated regions of adjacent endogenous genes face each other is not limited to the above sites, and may be another site where the 3′ side untranslated regions of adjacent endogenous genes face each other in the genomic DNA of cyanobacterium such as the Synechocystis sp. PCC 6803 strain.

The transfer site of the DNA fragment 25 in the target DNA is not limited to the site where 3′ side untranslated regions of adjacent endogenous genes face each other. For example, the transfer site of the DNA fragment in the target DNA may be a site where some or all of genes exhibiting a predetermined phenotype are deleted from the genomic DNA. In such a case, by observing the phenotype of cyanobacterium, the deletion of the genes can be confirmed to determine the success or failure of homologous recombination.

### (1-2) Selection Marker Region 21

The selection marker region 21 is a site for selecting a cyanobacterium in which homologous recombination has occurred. The selection marker region 21 has a gene that is transferred into the genomic DNA of cyanobacterium together with the DNA fragment 25 and functions as an index for selecting the cyanobacterium into which the DNA fragment 25 is transferred. Examples of such a gene include a drug resistance gene. Examples of the drug resistance gene include various antibiotic resistance genes such as a kanamycin resistance gene, a chloramphenicol resistance gene, and a hygromycin resistance gene. The selection marker region 21 may be removed after the DNA fragment 25 is transferred into the genomic DNA of cyanobacterium.

The selection marker region 21 preferably includes, together with the drug resistance gene, a transcriptional regulatory site and a transcription termination site for the drug resistance gene. According to such a configuration, it is possible to suppress the influence of the transcription of the drug resistance gene on the transcription of the genes present on the homologous recombination regions 11 and 12 adjacent to the selection marker region 21 and a foreign gene incorporated into the multi-cloning site 23. In addition, it is possible to suppress the influence of the transcription of the genes present on these homologous recombination regions 11 and 12 and the foreign gene on the transcription of the drug resistance gene.

### (1-3) Multi-Cloning Site 23

The multi-cloning site 23 has a restriction enzyme site for inserting the DNA fragment 25 between the first homologous recombination region 11 and the second homologous recombination region 12. According to such a configuration, the DNA fragment 25 is easily introduced into the gene transfer vector 10, which is preferable.

The multi-cloning site 23 may be constituted by a known sequence having a plurality of restriction enzyme sites. In the gene transfer vector 10, the type of restriction enzyme that can be used in cloning is limited depending on sequences of other regions of the gene transfer vector 10. The gene transfer vector 10 of the present disclosure has longer homologous recombination regions 11 and 12 than those of conventional vectors, and the type of restriction enzyme that can be used in cloning is also limited. The gene transfer vector 10 having the multi-cloning site 23 can be obtained by appropriately designing, for example, a combination of the type and number of restriction enzyme contained in the multi-cloning site 23 and the base sequences of the homologous recombination regions 11 and 12.

### (1-4) DNA Fragment 25

The DNA fragment 25 has, for example, any foreign gene intended to be transferred into the cyanobacterium. Examples of such a foreign gene can include a gene having a function of producing useful proteins, biofuels such as alcohols and oils, and substances such as chemical product alternative raw materials. In addition, as the foreign gene, a gene for improving the ability to produce useful substances by metabolic pathway improvement or the like can also be exemplified.

The length of the DNA fragment 25 is not particularly limited. The gene transfer vector 10 of the present disclosure has a property that, for example, the DNA fragment 25 of 5 kbp or more can be transferred into the genomic DNA of cyanobacterium, and thus is suitable for the transfer of a long DNA fragment 25. The present inventors have confirmed that a DNA fragment 25 of up to 35586 bp can be transferred into the genomic DNA of cyanobacterium using pFOSSynTSY17Km(p), which is an example of the gene transfer vector 10.

The phrase “a DNA fragment can be transferred” means that a transformant into which a DNA fragment is transferred can be obtained by a genetic engineering technique regardless of recombination efficiency. A length of the DNA fragment 25 that can be transferred using the gene transfer vector 10 may be 8 kbp or more, 10 kpb or more, 15 kpb or more, 20 kpb or more, 25 kpb or more, 30 kpb or more, or 35 kpb or more. An upper limit of the length of the DNA fragment 25 that can be transferred is not particularly limited, but may be 50 kbp or less or 40 kbp or less.

### (1-5) Other Areas

The gene transfer vector 10 may have other regions except the homologous recombination regions 11 and 12, the selection marker region 21, the multi-cloning site 23, and the DNA fragment 25. As such other regions, a region 27 for maintaining and replicating the gene transfer vector 10 in E. coli or the like can be exemplified (see FIG. 1).

### (1-6) Type of Vector

The gene transfer vector 10 is preferably a fosmid vector. The fosmid vector is a cloning vector based on a bacterial F-plasmid. Such a gene transfer vector 10 can be prepared using a known available fosmid vector. Examples of such a vector can include fosmid vectors such as pCC2FOS™ and pCC1FOS™. The gene transfer vector 10 is not limited to the fosmid vectors, and may be a cosmid vector, a viral vector, or the like.

The gene transfer vector 10 is preferably a fosmid vector having 1 copy per cell of E. coli. If ten to several hundred copies of plasmid or the like are used as a base per cell of E. coli, E. coli cannot be grown, so that the gene transfer vector 10 into which the DNA fragment 25 is introduced cannot be sufficiently obtained. By using a fosmid vector having a low copy number, the gene transfer vector 10 into which the DNA fragment 25 is introduced can be stably maintained in E. coli. As a result, the gene transfer vector 10 into which the DNA fragment 25 is introduced can be suitably obtained.

### (2) Transformant

The cyanobacterium according to the present embodiment is a transformant transformed using the gene transfer vector 10. By using the gene transfer vector 10, a stable transformant in which the DNA fragment 25 is incorporated into the genomic DNA can be easily and efficiently prepared. In addition, the cyanobacterium transformed using the gene transfer vector 10 has an advantageous feature that a total base length of regions unnecessary for production of a useful substance to be transferred into the genomic DNA (for example, the selection marker region and a region derived from the restriction enzyme site) is short, as compared with, for example, a cyanobacterium transformed a plurality of times using a vector capable of introducing only a DNA fragment of less than 5 kbp. Furthermore, the cyanobacterium transformed using the gene transfer vector 10 has an advantageous feature of a low risk of causing a mutation in DNA other than the transfer site, as compared with a cyanobacterium transformed a plurality of times as described above. That is, as the number of times of transformation increases, the risk of causing a mutation in DNA that is not originally desired to be damaged increases. However, the cyanobacterium of the present embodiment can be obtained through a small number of times of transformation, so that the mutation of DNA is suppressed.

### 2. Method for Producing Cyanobacterium

The method for producing a cyanobacterium of the present embodiment includes a step of transformation using a gene transfer vector. Specifically, the method for producing a cyanobacterium is a method including a step of constructing the gene transfer vector 10, and a step of introducing a DNA fragment 25 into the constructed gene transfer vector 10, and a step of obtaining a cyanobacterial transformant using the gene transfer vector 10 into which the DNA fragment 25 is introduced. Note that the above-described technique can be performed, for example, according to the description which will be described later, and more specifically, according to Examples which will be described later. The technique of each operation is not particularly limited, and various known genetic engineering techniques can be adopted.

### (1) Construction of Gene Transfer Vector 10

The gene transfer vector 10 can be constructed along the following operation procedures S1 to S5.
[S1] Extraction and Fragmentation of Genomic DNA of Cyanobacterium
[S2] Cloning

DNA fragments of about 25 to 50 kb are collected. Sequences of the DNA fragments are candidate for sequences of the homologous recombination regions. Hereinafter, the DNA fragments are also referred to as candidate DNA fragments.

Each of the collected candidate DNA fragments is inserted into a vector (for example, a fosmid vector) to prepare a vector (recombinant vector) having various candidate DNA fragments.
[S3] Preparation of E. coli (Transformant)

The vector having various candidate DNA fragments is introduced (packaged) into a bacteriophage.

A host such as E. coli is infected with the bacteriophage described above, and the vector is introduced to obtain E. coli (transformant) having the vector having various candidate DNA fragments.

The transformant is grown on an agar medium and obtained as colonies.
[S4] Selection of Candidate DNA Fragments

The vector is extracted from E. coli.

The base sequences of the candidate DNA fragments inserted into the vector are analyzed.

The analyzed base sequences are collated with the base sequences of the genomic DNA of cyanobacterium, and candidate DNA fragments having a restriction enzyme site cleavable (digestible) at one position are selected.
[S5] Addition of Selection Marker Region 21 and Multi-Cloning Site 23

A selection marker region 21 and a multi-cloning site 23 are added to the restriction enzyme sites in the candidate DNA fragments.

### (2) Construction of Gene Transfer Vector 10 Into Which DNA Fragment 25 is Introduced

The introduction (joint) of the DNA fragment 25 into (to) the gene transfer vector 10 can be performed according to the following operation procedures S6 to S8.
[S6] Provision of DNA Fragment 25

The DNA fragment 25 having a foreign gene of interest is increased by PCR or E. coli culture to adjust DNA fragment 25.
[S7] Provision of Vector DNA

The gene transfer vector 10 is cleaved at a restriction enzyme site on the multi-cloning site 23 to obtain a vector DNA.
[S8] Ligation of DNA Fragment 25 and Vector DNA

The adjusted DNA fragment 25 and the vector DNA are joined to obtain the gene transfer vector 10 into which the DNA fragment 25 is introduced. To join the vector DNA and the DNA fragment 25, a general ligation method (for example, use of enzyme ligase) or an In-Fusion system can be used.

### (3) Obtainment of Cyanobacterial Transformant

The transformation of cyanobacterium can be obtained according to the following operation procedures S9 to S10.
[S9] Preparation of Cyanobacterium (Transformant)

A cyanobacterium (wild-type strain, non-transformed strain) is mixed with the gene transfer vector 10 described above, and the gene transfer vector 10 is incorporated into cells of the cyanobacterium. The gene transfer vector incorporated into the cells causes homologous recombination with the target DNA on the genomic DNA, whereby the DNA fragment 25 is transferred between the 5′ side of the target DNA and the 3′ side of the target DNA (natural transformation).

At this time, a reagent such as EDTA may be used to increase the transformation efficiency.
[S10] Screening

The transformant is grown on an agar medium and obtained as colonies. At this time, the cyanobacterial transformant is selected using a selection marker.

### 3. Action/Effect of the Present Embodiment

According to the gene transfer vector 10 of the present embodiment, a long DNA fragment (for example, 5 kbp or more) can be transferred into the genomic DNA of cyanobacterium through one genetic recombination. Thus, the time taken to improve the cyanobacterium can be significantly reduced.

The literature “Scientific Reports, Vol. 8, Article number: 7380 (2018)” reports the following technique as a technique for transferring a DNA fragment of about 20 kbp into a genomic DNA of cyanobacterium. In this technique, the DNA fragment of about 20 kbp is divided into about 4 kbp pieces, which are transfected into the genomic DNA by performing genetic recombination and selection marker removal operations five times. In the selection marker removal, a system called SacB is used to remove antibiotic resistance genes from the genomic DNA. This technique requires a long time to obtain a cyanobacterial transformant into which a long DNA fragment 25 is transferred. On the other hand, by using the gene transfer vector 10 of the present disclosure, a cyanobacterial transformant into which the long DNA fragment 25 is transferred can be obtained through a small number (for example, one) of gene recombination operations. In addition, by using the gene transfer vector 10 of the present disclosure, the number of times of the operation for removing the selection marker region 21 or the like can be reduced. As a result, the target cyanobacterial transformant can be obtained in a short period of time.

The mechanism by which the long DNA fragment 25 can be transferred using the gene transfer vector 10 of the present disclosure is not clear, but is presumed as follows. Note that the present disclosure is not to be construed as being limited by the presumed reason.

Conventionally, it has not been easy to synthesize a vector having long homologous recombination regions. For this reason, due to insufficient lengths of the homologous recombination regions, a long DNA fragment could not be transferred into the genomic DNA of cyanobacterium. On the other hand, it is presumed that, since the gene transfer vector 10 of the present disclosure has a homologous recombination region of 5 kbp or more, even a long DNA fragment 25 can be transferred into the genomic DNA of cyanobacterium.

### EXAMPLE

Hereinafter, the present invention will be described in more detail based on Examples, but is not limited thereto.

### 1. Construction of Gene Transfer Vector

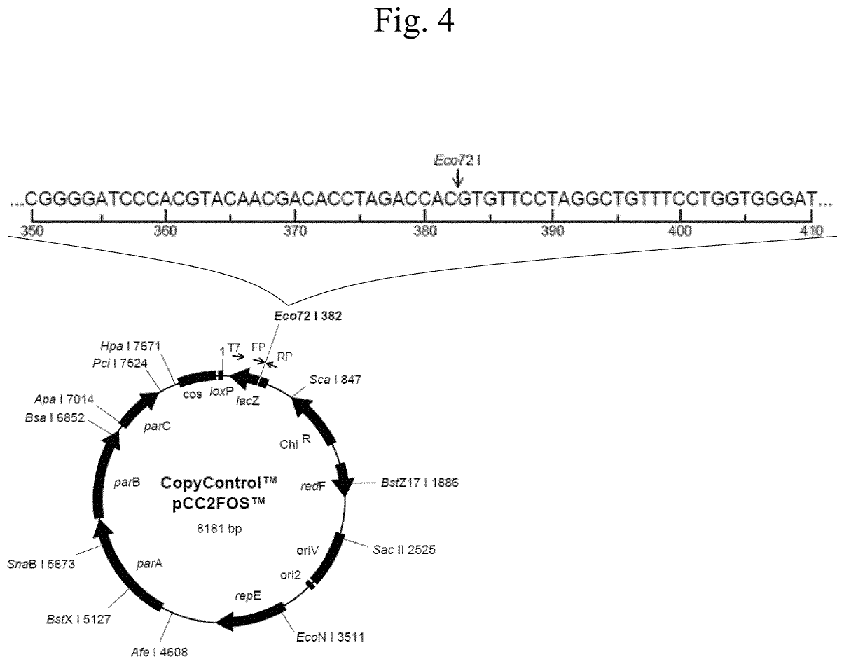

The genomic DNA extracted from cyanobacterium Synechocystis sp. PCC 6803 was physically sheared, specifically, an extracted genomic DNA solution was drawn up and dispensed five times by a fine-tipped pipette tip (manufactured by QSP), and blunt ends were generated by End-Repair Enzyme Mix (manufactured by Epicentre). Alternatively, vortex or sonication, etc., may be used for the physical shearing. The blunt-ended DNA fragments were sorted by size using pulse field electrophoresis, and the DNA fragments having an average strand length of 25 to 40 kb were collected. The collected DNA fragments were purified with NucleoSpin gDNA Clean-up (MACHEREY-NAGEL GmbH & Co KG). Next, the purified DNA fragments were ligated to Eco72I site (between 382nd C and 383rd G, FIG. 4) of CopyControl™, pCC2FOS™ Fosmid Vectors (manufactured by Epicentre) having a chloramphenicol resistance gene. T4DNA ligase (manufactured by TaKaRa) was used for ligation to the fosmid vectors. MaxPlax™ Lambda Packaging Extracts (manufactured by Epicentre) were used for in vitro packaging of the DNA ligated to the fosmid vectors. They were infected with E. coli EPI-300T1R (manufactured by Epicentre) (hereinafter, referred to as EPI300), which is T1 phage resistant E. coli. Then, about 2.0×10⁵ transformed E. coli (transformant) was obtained in LB medium (LB/Cm) agar plates containing 12.5 μg/mL of chloramphenicol. About 100 colonies were taken from the transformed E. coli, and each was cultured in 4 mL of LB medium (LB/Cm) containing 12.5 μg/mL of chloramphenicol, in the air at 37° C. and 180 rpm for 18 hours. Then, the cultured E. coli was collected, and fosmid DNA introduced into E. coli was extracted with Wizard Plus SV Minipreps DNA Purification Systems (manufactured by Promega). Next, the base sequences of DNA derived from the cyanobacterium Synechocystis cloned into the extracted fosmid DNA were analyzed. The base sequences were analyzed using primers pCC2 forward-b and pCC2 reverse-b. The base sequences of the primers used are as follows.

192_pCC2 forward_b,
(SEQ ID NO: 7)
CCAGTCACGACGTTGTAAAACG
194_pCC2 reverse_b,
(SEQ ID NO: 8)
CGCCAAGCTATTTAGGTGAGAC

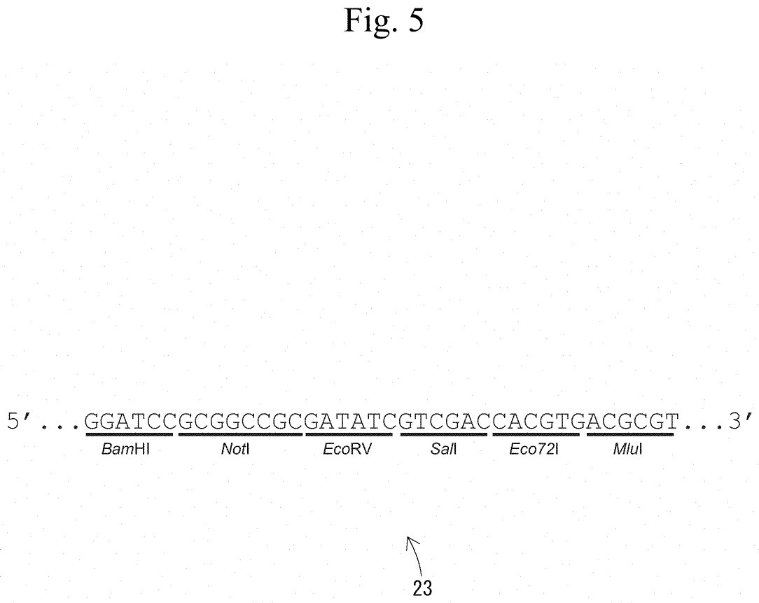

The analyzed base sequences were collated with the base sequences of the genomic DNA of cyanobacterium Synechocystis (using CyanoBase ([genome.microbedb.jp/cyanobase/]) or KEGG, Kyoto Encyclopedia of Genes and Genomes ([www.genome.jp/kegg/])) to obtain information on all the base sequences (about 20 to 40 kbp) cloned into the fosmid DNA. From these base sequences, fosmids having a restriction enzyme site cleavable (digestible) at one position around the center of the DNA fragment were selected. A cassette of kanamycin resistance gene (SEQ ID NO: 5) and cloning sites (BamHI, NotI, EcoRV, SalI, Eco72I, and MluI) (FIG. 5) were added to this restriction enzyme site (in the Example, SmiI site), and a DNA fragment was partially added by PCR so as not to damage the genes originally possessed by the cyanobacterium Synechocystis. The synthesized DNA was designated as pFOSSynTSY17Km(p) (an example of the gene transfer vector, FIG. 2).

The gene transfer site TSY17 in the case of using pFOSSynTSY17Km(p) is a site where the 3′ side untranslated regions of the slr 1716 gene and the sll 1609 gene on the genomic DNA of the cyanobacterium Synechocystis face each other. In pFOSSynTSY17Km(p), the length of the first homologous recombination region (SEQ ID NO: 1) was 18178 bp and the length of the second homologous recombination region (SEQ ID NO: 2) was 19602 bp. The total length of these homologous recombination regions was 37.8 kbp.

The gene transfer site TSY21 in the case of using pFOSSynTSY21Km(p) (another example of the gene transfer vector, FIG. 3) obtained by a similar method is a site where the 3′ side untranslated regions of the slr 1966 gene and the sll 1893 gene on the genomic DNA of the cyanobacterium Synechocystis face each other. In pFOSSynTSY21Km(p), the length of the first homologous recombination region (SEQ ID NO: 3) was 24303 bp and the length of the second homologous recombination region (SEQ ID NO: 4) was 13934 bp. The total length of these homologous recombination regions was 38.2 kbp.

### 2. Construction of Gene Transfer Vector Having DNA Fragment Introduced Thereinto

pFOSSynTSY17Km(p) was cleaved (digested) with the restriction enzyme Eco72I, blunt ends were generated by End-Repair Enzyme Mix (manufactured by Epicentre), and the blunt-ended product was used as a vector DNA. Next, a DNA of about 17.5 kbp (SEQ ID NO: 6) containing a human β-globin gene (hβG) was PCR amplified using human genomic DNA (manufactured by Promega) as a template DNA and KOD-plus-neo polymerase (manufactured by TOYOBO). The base sequences of the primers used are as follows.

444_hGB17.5F-IFSE2,
(SEQ ID NO: 9)
GATATCGTCGACCACTGCACCTGCTCTGTGATTATGACTA
TCC
445_hGB17.5R-IFAN2,
(SEQ ID NO: 10)
TCTAGAACGCGTCACACATGATTAGCAAAAGGGCCTAGCT
TG

The obtained DNA fragment was used as an insert DNA.

The vector DNA and the insert DNA were ligated using In-Fusion (manufactured by TaKaRa) to synthesize pFOSSynTSY17Km(p)-hβG DNA in which a 17.5 kbp human β-globin gene (hβG) was introduced into the Eco72I site of pFOSSynTSY17Km(p).

### 3. Transformation of Cyanobacterium Synechocystis

The cyanobacterium Synechocystis sp. PCC 6803 strain was used (hereinafter, referred to as Synechocystis). Synechocystis was aeration-cultured in BG11 medium containing 20 mM TES-KOH (pH 7.5) as a basic medium under continuous irradiation with white light (50 μmol of photon m⁻² s⁻¹) at 25° C. with stirring using a rotary shaker. In the case of a solid medium, a medium to which 1.5% Agar (manufactured by Wako) was added was used. Cell turbidity of Synechocystis was evaluated by measuring optical turbidity (OD 730) using a spectrophotometer (Bio-Spec mini., manufactured by Shimadzu).

One (1) μg of pFOSSynTSY17Km(p)-hβG DNA was added to cells of 40 μL (OD 730=25) of a culture solution of cyanobacterium Synechocystis. The cells were statically cultured at 25° C. for 4 hours in the dark, and spread on BG11 agar medium containing 20 μg/ml of the antibiotic kanamycin. The cells were cultured at 25° C. for 10 days in a bright place (50 μmol of photon m⁻² s⁻¹) to obtain a plurality of colonies exhibiting kanamycin resistance.

### 4. Confirmation of Introduced DNA of Transformant

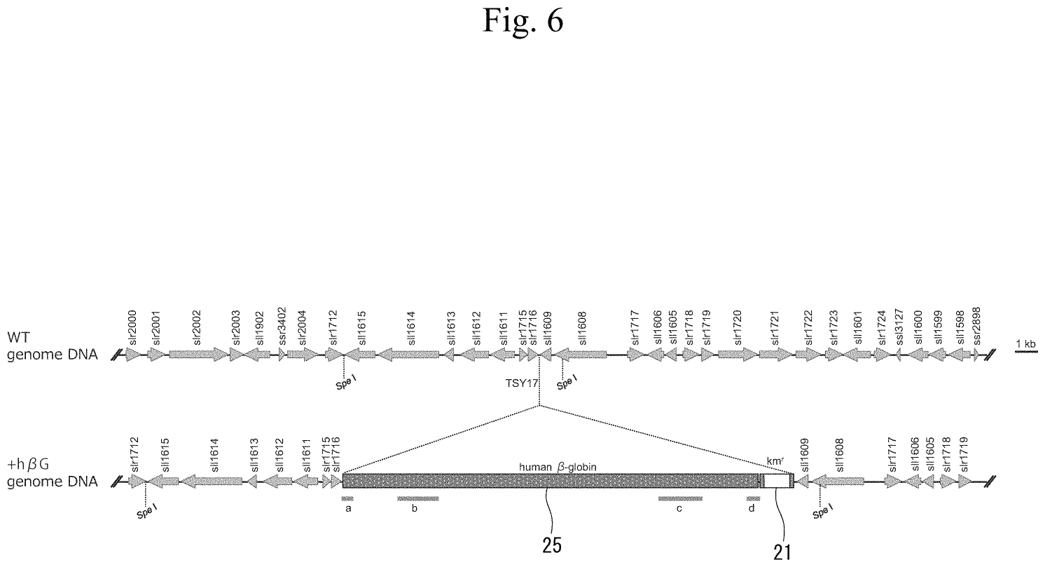

The obtained colonies were cultured in BG11 liquid medium containing kanamycin, and genomic DNA was extracted. Insertion of the introduced DNA (β-globin gene, about 17.5 kbp) into the extracted genomic DNA was confirmed by PCR and Southern blotting. FIG. 6 shows the constitution of the genomic DNA of the cyanobacterium Synechocystis. In FIG. 6, “WT” represents a wild-type strain, and “+hβG” represents a transformed strain.

### (1) PCR

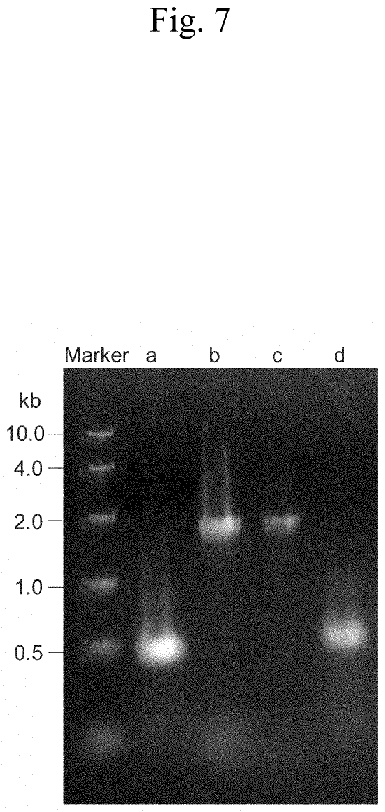

Using the extracted genomic DNA as a template, the introduced DNA was PCR amplified with KOD-plus-neo polymerase (manufactured by TOYOBO) using the following primers. Bands a to d of DNAs derived from the introduced DNA (β-globin gene) shown in FIG. 6 were confirmed by agarose electrophoresis (see FIG. 7). The base sequences of the used primers and the size of the detected DNA are as follows.

Detection of band a in FIG. 7 (462 bp)
390_hGB17.5F,
(SEQ ID NO: 11)
TGCACCTGCTCTGTGATTATGACTATCCCACAGTC
402_hGB17.5F-seq-AN,
(SEQ ID NO: 12)
AGTGGCCTTCCATTATTCATAGTCCTTGCTCTA
CC
Detection of band b in FIG. 7 (1,728 bp)
674_hBG-check1-SE,
(SEQ ID NO: 13)
GAAACTGGATGCAGAGACCAGATG
675_hBG-check1-An,
(SEQ ID NO: 14)
AACTATAGCAGAGGCAGAGGAAGG
Detection of band c in FIG. 7 (1, 798 bp)
676_hBG-check2-SE,
(SEQ ID NO: 15)
ACCACACTCCCATAGATGAGTGTC
677_hBG-check2-An,
(SEQ ID NO: 16)
CTCTATAGCTTCCCAACGTGATCG
Detection of band d in FIG. 7 (509 bp)
403_hGB17.5R-seq-SE,
(SEQ ID NO: 17)
CAGTCTGCCTAGTACATTACTATTTGGAATATA
TG
391_hGB17.5R,
(SEQ ID NO: 18)
ACATGATTAGCAAAAGGGCCTAGCTTGGACTCAGA

### (2) Southern Blotting

The extracted genomic DNA was digested (37° C., 2 h) with the restriction enzyme SpeI (manufactured by TaKaRa). The digested sample was subjected to electrophoresis, then transferred to a nylon membrane, and a probe labeled with PCR DIG Probe Synthesis Kit (manufactured by Sigma-Aldrich) was hybridized. For the synthesis of the probe, the 462 bp DNA fragment PCR amplified with the following primers was used.

390_hGB17.5F,
(SEQ ID NO: 11)
TGCACCTGCTCTGTGATTATGACTATCCCACAGTC
402_hGB17.5F-seq-AN,
(SEQ ID NO: 12)
AGTGGCCTTCCATTATTCATAGTCCTTGCTCTA
CC

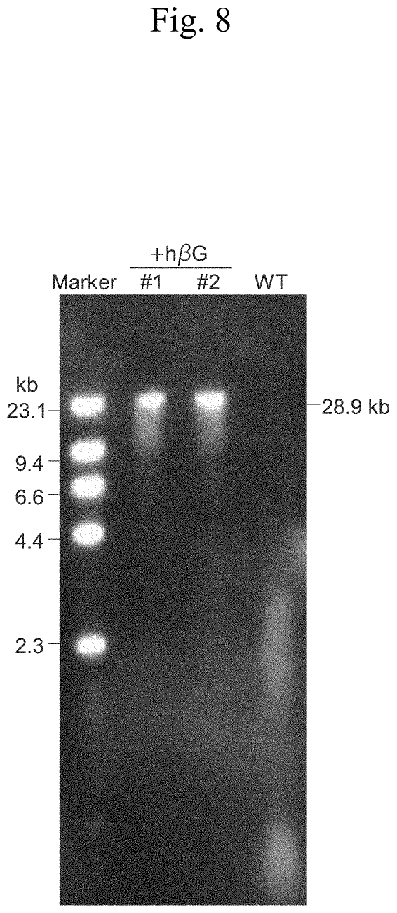

The hybridized membrane was immersed in CDP-Star, and the signal of the probe was detected with Fusion FX Chemiluminescence Imaging System (manufactured by Vilber-Lourmat). As a result, a band of 28.9 kbp (28,862 bp) including the human β-globin gene 17.5 kbp (17,567 bp) was detected (FIG. 8). It was confirmed that a gene of up to 35.6 kbp (35,586 bp) can be transferred into the genomic DNA of the cyanobacterium Synechocystis by the same method.

### 5. Effect of Example

Using the gene transfer vector of the Example, a long DNA fragment could be transferred into the genomic DNA of cyanobacterium to provide a useful cyanobacterium.

The present invention is not limited to the above embodiment and example, and various modifications can be made.

What is claimed is:

1. A cyanobacterium transformed using a gene transfer vector,
the gene transfer vector comprising:
a first homologous recombination region homologous to a 5′ side of a target DNA of a cyanobacterium; a second homologous recombination region homologous to a 3′ side of the target DNA; and a DNA fragment introduced into a portion sandwiched between the first homologous recombination region and the second homologous recombination region,
wherein a total length of the first homologous recombination region and the second homologous recombination region is 5 kbp or more, and
wherein the DNA fragment is transferred between the 5′ side of the target DNA and the 3′ side of the target DNA.

2. The cyanobacterium according to claim 1, wherein a transfer site of the DNA fragment in the target DNA is a site where 3′ side untranslated regions of adjacent endogenous genes face each other.

3. The cyanobacterium according to claim 2, wherein the transfer site of the DNA fragment in the target DNA is a site where 3′ side untranslated regions of an slr 1716 gene and an sll 1609 gene in a cyanobacterium Synechocystis sp. PCC 6803 strain face each other.

4. The cyanobacterium according to claim 2, wherein the transfer site of the DNA fragment in the target DNA is a site where 3′ side untranslated regions of an slr 1966 gene and an sll 1893 gene in a cyanobacterium Synechocystis sp. PCC 6803 strain face each other.

5. The cyanobacterium according to claim 1, wherein the DNA fragment is 5 kbp or more.

6. A method for producing a cyanobacterium comprising transforming the cyanobacterium using a gene transfer vector,
the gene transfer vector comprising:
a first homologous recombination region homologous to a 5′ side of a target DNA of a cyanobacterium; a second homologous recombination region homologous to a 3′ side of the target DNA; and a DNA fragment introduced into a portion sandwiched between the first homologous recombination region and the second homologous recombination region,
wherein a total length of the first homologous recombination region and the second homologous recombination region is 5 kbp or more, and
wherein, in the transforming, the DNA fragment is transferred between the 5′ side of the target DNA and the 3′ side of the target DNA.

7. The method for producing a cyanobacterium according to claim 6, wherein a transfer site of the DNA fragment in the target DNA is a site where 3′ side untranslated regions of adjacent endogenous genes face each other.

8. The method for producing a cyanobacterium according to claim 7, wherein the transfer site of the DNA fragment in the target DNA is a site where 3′ side untranslated regions of an slr 1716 gene and an sll 1609 gene in a cyanobacterium Synechocystis sp. PCC 6803 strain face each other.

9. The method for producing a cyanobacterium according to claim 7, wherein the transfer site of the DNA fragment in the target DNA is a site where 3′ side untranslated regions of an slr 1966 gene and an sll 1893 gene in a cyanobacterium Synechocystis sp. PCC 6803 strain face each other.

10. The method for producing a cyanobacterium according to claim 6, wherein the DNA fragment is 5 kbp or more.

11. A gene transfer vector used for a cyanobacterium, comprising:
a first homologous recombination region homologous to a 5′ side of a target DNA of the cyanobacterium; and a second homologous recombination region homologous to a 3′ side of the target DNA,
wherein a total length of the first homologous recombination region and the second homologous recombination region is 5 kbp or more.

12. The gene transfer vector according to claim 11, which has a selection marker region in a portion sandwiched between the first homologous recombination region and the second homologous recombination region.

13. The gene transfer vector according to claim 11, which has a multi-cloning site for introducing a DNA fragment in a portion sandwiched between the first homologous recombination region and the second homologous recombination region.

14. The gene transfer vector according to claim 11, which is a fosmid vector.
